SEQUENCE LISTING (12 sequences)

SEQ ID NO: 1 - DNA, 10 bp, synthetic construct
  misc_feature 1..10: note = Synthetic Polynucleotide
  source 1..10: mol_type = other DNA
tcaacgttga

SEQ ID NO: 2 - DNA, 20 bp, synthetic construct
  misc_feature 1..20: note = Synthetic Polynucleotide
  misc_feature 20: note = (Sp18)2-chol
  source 1..20: mol_type = other DNA
ggggtcaacgttgagggggg

SEQ ID NO: 3 - DNA, 20 bp, synthetic construct
  misc_feature 1..20: note = Synthetic Polynucleotide
  misc_feature 20: note = (Sp18)2-chol
  source 1..20: mol_type = other DNA
tccatgacgttcctgacgtt

SEQ ID NO: 4 - DNA, 20 bp, synthetic construct
  misc_feature 1..20: note = Synthetic Polynucleotide
  source 1..20: mol_type = other DNA
ggggtcaacgttgagggggg

SEQ ID NO: 5 - DNA, 20 bp, synthetic construct
  misc_feature 1..20: note = Synthetic Polynucleotide
  source 1..20: mol_type = other DNA
tccatgacgttcctgacgtt

SEQ ID NO: 6 - DNA, 20 bp, synthetic construct
  misc_feature 1..20: note = Synthetic Polynucleotide
  misc_feature 20: note = (Sp18)-FAM-(Sp18)-chol
  source 1..20: mol_type = other DNA
ggggtcaacgttgagggggg

SEQ ID NO: 7 - DNA, 20 bp, synthetic construct
  misc_feature 1..20: note = Synthetic Polynucleotide
  misc_feature 20: note = (Sp18)-Cy3-(Sp18)-chol
  source 1..20: mol_type = other DNA
tccatgacgttcctgacgtt

SEQ ID NO: 8 - DNA, 20 bp, synthetic construct
  misc_feature 1..20: note = Synthetic Polynucleotide
  misc_feature 20: note = (Sp18)-Cy5-(Sp18)-chol
  source 1..20: mol_type = other DNA
tccatgacgttcctgacgtt

SEQ ID NO: 9 - DNA, 20 bp, synthetic construct
  misc_feature 1..20: note = Synthetic Polynucleotide
  misc_feature 20: note = FAM
  source 1..20: mol_type = other DNA
ggggtcaacgttgagggggg

SEQ ID NO: 10 - DNA, 20 bp, synthetic construct
  misc_feature 1..20: note = Synthetic Polynucleotide
  misc_feature 20: note = Cy5
  source 1..20: mol_type = other DNA
tccatgacgttcctgacgtt

SEQ ID NO: 11 - DNA, 20 bp, Homo sapiens
  source 1..20: mol_type = genomic DNA
gggggacgatcgtcgggggg

SEQ ID NO: 12 - DNA, 24 bp, Homo sapiens
  source 1..24: mol_type = genomic DNA
tcgtcgttttgtcgttttgtcgtt

(19) United States
(12) Patent Application Publication
(10) Pub. No.: US 2025/0075219 A1
(43) Pub. Date: Mar. 6, 2025

(54) Sequence Multiplicity within Spherical Nucleic Acids

(71) Applicant: NORTHWESTERN UNIVERSITY, Evanston, IL (US)

(72) Inventors: Chad A. Mirkin, Wilmette, IL (US); Ziyin Huang, Evanston, IL (US)

(21) Appl. No.: 18/949,786

(22) Filed: Nov. 15, 2024

Related U.S. Application Data
(63) Continuation of application No. 17/084,460, filed on Oct. 29, 2020.
(60) Provisional application No. 62/927,456, filed on Oct. 29, 2019.

(51) Int. Cl. C12N 15/117; A61K 31/7088; A61K 31/7125; A61K 47/69

(52) U.S. Cl. CPC C12N 15/117; A61K 31/7088; A61K 31/7125; A61K 47/6911; C12N 2310/17; C12N 2310/315; C12N 2320/32; C12Q 2525/307

(57) ABSTRACT

The present disclosure is directed to spherical nucleic acids (SNAs) comprising a nanoparticle core and an oligonucleotide shell attached to the external surface of the nanoparticle core, wherein the oligonucleotide shell comprises a mixture of class A CpG oligonucleotides and class B CpG oligonucleotides. The disclosure also provides methods of using the SNAs for, e.g., regulation of an immune response and gene regulation.

### CROSS-REFERENCE TO RELATED APPLICATIONS

This application is a Continuation of U.S. application Ser. No. 17/084,460, filed Oct. 29, 2020, which claims the priority benefit under 35 U.S.C. §119(e) of U.S. Provisional Patent Application No. 62/927,456, filed Oct. 29, 2019, which are incorporated herein by reference in their entirety.

### STATEMENT OF GOVERNMENT INTEREST

This invention was made with government support under grant numbers U54CA 199091-01 and R01CA208783-01 awarded by the National Institutes of Health. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “2019-195A_Seqlisting.txt”, which was created on Nov. 15, 2024 and is 18,072 bytes in size. The subject matter of the Sequence Listing is incorporated herein in its entirety by reference.

### BACKGROUND

Spherical nucleic acids (SNAs) are a class of nanostructures consisting of a nanoparticle core surrounded by a shell of highly oriented and densely packed oligonucleotides.¹⁻² Such structures are typically more stable than comparable linear strands in biological milieu and exhibit high cellular internalization via scavenger receptor-mediated endocytosis.³ SNAs made with liposome cores, termed liposomal SNAs (LSNAs), are biocompatible and suitable for a wide variety of biomedical applications.⁴⁻⁹ To date, with the exception of the use of secondary strands as diluents,¹⁰ material-directing units,¹¹⁻¹² or probe entities,¹³⁻¹⁵ SNAs have been prepared exclusively from a single oligonucleotide sequence.

### SUMMARY

Delivering multiple oligonucleotide sequences (e.g., DNA, RNA, etc.) to the same target cell is beneficial in many applications, but direct delivery cannot ensure co-delivery. It is shown herein that multiple oligonucleotide sequences on one spherical nucleic acid (SNA), which is a nanostructure comprising a nanoparticle core surrounded by a shell of highly oriented and densely packed oligonucleotides, enabled efficient co-delivery of several distinct oligonucleotide sequences to the same cell at the pre-determined desired ratios of these sequences.

Thus, the present disclosure is generally directed to spherical nucleic acids (SNAs) comprising a nanoparticle core and an oligonucleotide shell attached to the external surface of the nanoparticle core, wherein the oligonucleotide shell comprises a mixture of oligonucleotides.

Applications of the technology disclosed herein include, but are not limited to: nucleic acid-based therapy, combination therapy, and cancer immunotherapy. Advantages of the technology disclosed herein include, but are not limited to: co-delivery of multiple oligonucleotide sequences to the same cell at controlled ratios for enhanced/synergistic responses; physically (e.g., structural features) and chemically (e.g., backbone chemistry) distinct oligonucleotide sequences.

Accordingly, in some aspects the disclosure provides a spherical nucleic acid (SNA) comprising a nanoparticle core and an oligonucleotide shell attached to the external surface of the nanoparticle core, wherein the oligonucleotide shell comprises a mixture of class A CpG oligonucleotides and class B CpG oligonucleotides, wherein the SNA does not comprise a class C CpG oligonucleotide. In some aspects, the disclosure provides a spherical nucleic acid (SNA) comprising a nanoparticle core and an oligonucleotide shell attached to the external surface of the nanoparticle core, wherein the oligonucleotide shell consists of a mixture of class A CpG oligonucleotides and class B CpG oligonucleotides. In further aspects, the disclosure provides a spherical nucleic acid (SNA) comprising a nanoparticle core and an oligonucleotide shell attached to the external surface of the nanoparticle core, wherein the oligonucleotide shell comprises a mixture of class A CpG oligonucleotides and class B CpG oligonucleotides, wherein the ratio of class A CpG oligonucleotides to class B CpG oligonucleotides in the mixture is about 4:1 to about 1:1. In some embodiments, the class A CpG oligonucleotides each comprise (i) an internal palindrome sequence containing a CpG motif and (ii) an at least partially phosphorothioated poly(G) sequence at its 5′ and/or 3′ ends. In some embodiments, the class A CpG oligonucleotide is DNA. In further embodiments, the class B CpG oligonucleotides each comprise a fully phosphorothioated sequence comprising one or more CpG motifs. In some embodiments, the class B CpG oligonucleotide is DNA. In some embodiments, the oligonucleotide shell comprises about 5 to about 150 oligonucleotides. In further embodiments, the oligonucleotide shell comprises about 5 to about 50 oligonucleotides. In further embodiments, the oligonucleotide shell comprises about 75 to about 100 oligonucleotides. In some embodiments, the ratio of class A CpG oligonucleotides to class B CpG oligonucleotides in the mixture is about 7:3. In further embodiments, the ratio of class A CpG oligonucleotides to class B CpG oligonucleotides in the mixture is about 4:1 to about 1:1. In some embodiments, each class A CpG oligonucleotide is about 10 to about 50 nucleotides in length. In further embodiments, each class A CpG oligonucleotide is about 15 to about 26 nucleotides in length. In some embodiments, each class B CpG oligonucleotide is about 10 to about 50 nucleotides in length. In some embodiments, each class B CpG oligonucleotide is about 18 to about 28 nucleotides in length. In some embodiments, a SNA of the disclosure further comprises an inhibitory oligonucleotide. In further embodiments, the inhibitory oligonucleotide is an antagonist oligonucleotide, antisense DNA, small interfering RNA (siRNA), an aptamer, a short hairpin RNA (shRNA), a DNAzyme, or an aptazyme. In some embodiments, the nanoparticle core is a metallic core, a semiconductor core, an insulator core, an upconverting core, a micellar core, a dendrimer core, a liposomal core, a polymer core, a metal-organic framework core, a protein core, or a combination thereof. In some embodiments, the polymer is polylactide, a polylactide-polyglycolide copolymer, a polycaprolactone, a polyacrylate, alginate, albumin, polypyrrole, polythiophene, polyaniline, polyethylenimine, poly (methyl methacrylate), poly (lactic-co-glycolic acid) (PLGA), or chitosan. In some embodiments, the nanoparticle core is gold, silver, platinum, aluminum, palladium, copper, cobalt, indium, cadmium selenide, iron oxide, fullerene, metal-organic framework, silica, zinc sulfide, or nickel. In some embodiments, the nanoparticle core is a protein core. In further embodiments, the protein core is an enzyme, a therapeutic protein, a structural protein, a defensive protein, a storage protein, a transport protein, a hormone, a receptor protein, a motor protein, or a fluorescent protein. In some embodiments, a SNA of the disclosure further comprises an additional agent. In further embodiments, the additional agent is a protein, a small molecule, or a peptide, or a combination thereof. In some embodiments, the protein is an antibody, an antigen, a cytokine, a chemokine, an interferon, or a combination thereof. In some embodiments, the SNA is from about 1 to about 150 nanometers (nm) in diameter. In some embodiments, the oligonucleotide shell comprises about 4 to about 1000 oligonucleotides.

In some aspects, the disclosure provides a composition comprising a plurality of spherical nucleic acids (SNAs) of the disclosure.

In further aspects, the disclosure provides a method of inhibiting expression of a gene comprising the step of hybridizing a polynucleotide encoding the gene product with a spherical nucleic acid (SNA) or composition of the disclosure, wherein hybridizing between the polynucleotide and one or more oligonucleotides in the oligonucleotide shell occurs over a length of the polynucleotide with a degree of complementarity sufficient to inhibit expression of the gene product. In some embodiments, expression of the gene product is inhibited in vivo. In some embodiments, expression of the gene product is inhibited in vitro.

In some aspects, the disclosure provides a method for up-regulating activity of a toll-like receptor (TLR), comprising contacting a cell having the toll-like receptor with a spherical nucleic acid (SNA) or composition of the disclosure. In some embodiments, the oligonucleotide shell further comprises one or more oligonucleotides that is a TLR agonist. In various embodiments, the toll-like receptor is chosen from the group consisting of toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, and toll-like receptor 13.

In some aspects, the disclosure provides a method for down-regulating activity of a toll-like receptor (TLR), comprising contacting a cell having the toll-like receptor with a spherical nucleic acid (SNA) or composition of the disclosure. In some embodiments, the oligonucleotide shell further comprises one or more oligonucleotides that is a TLR antagonist. In further embodiments, the toll-like receptor is chosen from the group consisting of toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, and toll-like receptor 13. In some embodiments, the method is performed in vitro. In some embodiments, the method is performed in vivo.

In some aspects, the disclosure provides a method of treating a disorder comprising administering an effective amount of a SNA or composition of the disclosure to a subject in need thereof, wherein the administering treats the disorder. In some embodiments, the disorder is cancer, an infectious disease, an autoimmune disease, or a combination thereof.

### BRIEF DESCRIPTION OF THE DRAWINGS

FIG. 1 depicts SNA designs with sequence multiplicity. Upper case letters=nucleobases with PO backbone; lower case letters=nucleobases with PS backbone; chol=cholesterol anchor.

FIG. 2 shows the characterization of dual-CpG SNAs (50:50 mol ratio CpG-A to CpG-B). (a) FRET efficiency comparison between FAM-CpG-A SNA/Cy3-CpG-B SNA mixture and dual-CpG SNA. Fluorescence signals of (b) FAM-CpG-A and (c) Cy5-CpG-B after 30-minute treatments in single-component or dual-CpG form. (d) Pearson correlation coefficient (p) between CpG-A and CpG-B uptake in dual-CpG SNA and linear CpG mixture forms. Representative flow cytometry data of (e) dual-CpG SNA and (f) linear CpG mixture uptake. (g) Manders overlap coefficients between CpG-A and CpG-B in dual-CpG SNA and linear CpG mixture forms over time after cellular uptake. (h) Representative confocal microscopy images of dual-CpG SNA 30 minutes, 4 hours, and 24 hours after initial uptake. Color assignments: FAM-CpG-A (green), Cy5-CpG-B (red), nucleus (blue), actin (gray). All dual-CpG data shown in this figure incorporated a 1:1 ratio of CpG-A: CpG-B.

FIG. 3 shows a CryoTEM image of LSNAs functionalized with 75 equivalents of class B CpG oligodeoxynucleotide (ODN). Scale bar: 100 nm.

FIG. 4 shows the characterization of CpG-A SNAs. (a) Agarose gel electrophoresis of liposomes functionalized with different amounts of cholesterol-labeled CpG-A ODN; the symbol ∞ indicates free cholesterol-labeled CpG-A ODN. (b) Hydrodynamic size of CpG-A SNAs as measured by DLS. CryoTEM images of: (c) bare liposomes and (d) SNAs functionalized with 150 CpG-A ODN. Scale bars: 100 nm.

FIG. 5 shows a CryoTEM image of LSNAs functionalized with 50 equivalents of class A CpG ODN. Scale bar: 100 nm.

FIG. 6 shows an agarose gel showing CpG components in dual-CpG SNAs. Lanes from left to right: 1: free CpG-A, 2: CpG-A SNA, 3˜7: 10%, 30%, 50%, 70%, and 90% dual-CpG SNAs, 8: CpG-B SNA, 9: free CpG-B.

FIG. 7 shows representative flow cytometry data of SNA and linear CpG mixture uptake after 30 minutes of treatment.

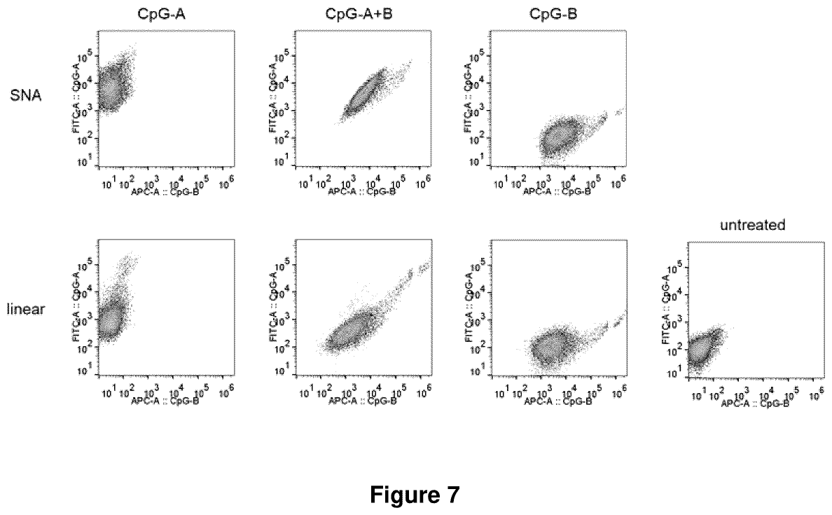

FIG. 8 shows representative confocal microscopy images of linear CpG mixture and dual-CpG SNA at different time points. Color assignments: FAM-CpG-A (green), Cy5-CpG-B (red), nucleus (blue), actin (gray).

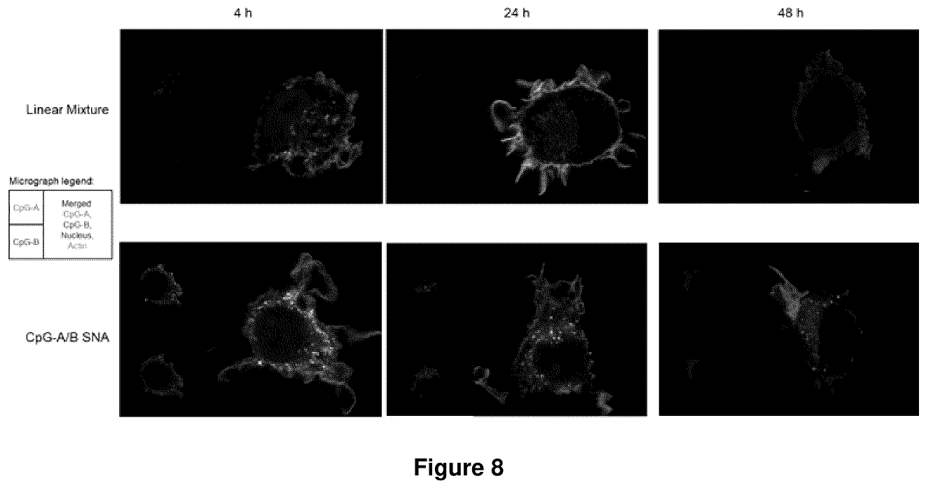

FIG. 9 shows cytokine and IFN production following intracellular localization. Production of (a) TNF-α, (b) IL-1α, and (c) IFN-α (^{#}signal below the lower limit of detection for untreated samples). Manders overlap coefficient of (d) CpG-SNAs or (e) CpG ODN in intracellular markers over time. Dual-CpG data shown in (d) and (e) incorporated a 1:1 ratio of CpG-A to CpG-B.

FIG. 10 shows IL-1β, IL-12p70, and IL-6 production.

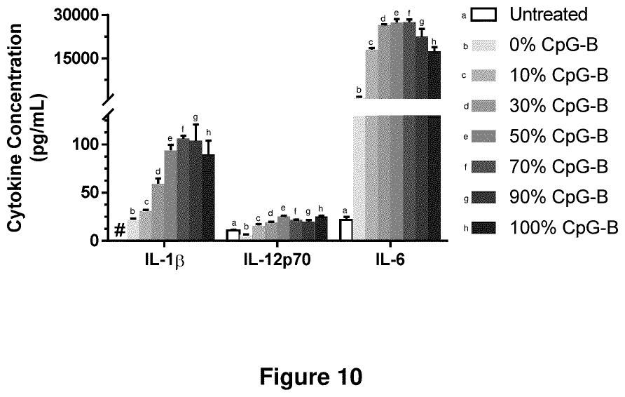

FIG. 11 shows confocal images of JAWS II cells treated with (a) CpG SNAs or (b) linear CpG ODNs and their relative localization in early endosomes. Color assignments: FAM-CpG-A (green), Cy5-CpG-B (red), early endosome (purple), nucleus (blue).

FIG. 12 shows confocal images of JAWS II cells treated with (a) CpG SNAs or (b) linear CpG ODNs and their relative localization in late endosomes. Color assignments: FAM-CpG-A (green), Cy5-CpG-B (red), late endosome (purple), nucleus (blue).

FIG. 13 shows confocal images of JAWS Il cells treated with (a) CpG SNAs or (b) linear CpG ODNs and their relative localization in lysosomes. Color assignments: FAM-CpG-A (green), Cy5-CpG-B (red), lysosome (purple), nucleus (blue).

FIG. 14 shows Manders overlap coefficient of dual-CpG in intracellular markers over time as shown in FIG. 9d-e, with CpG-A and CpG-B coefficients plotted separately.

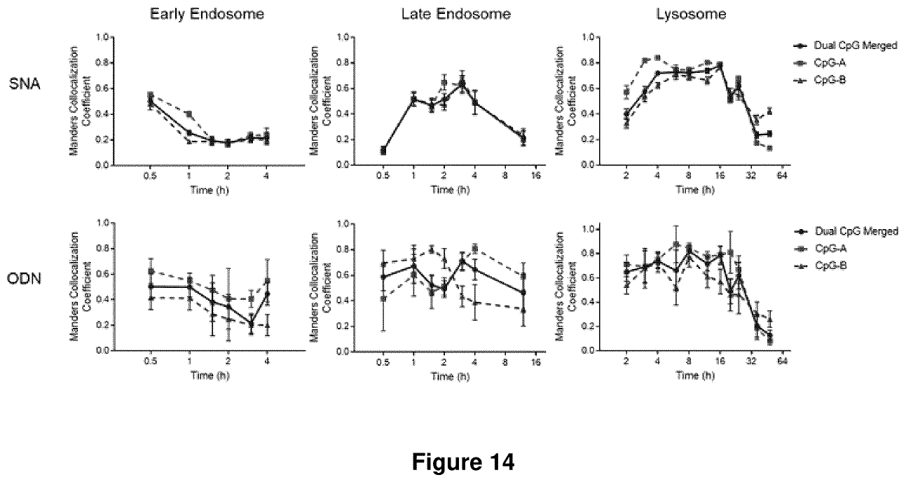

FIG. 15 shows DC costimulation marker activation. (a) Representative flow cytometry data for CD80 and CD86 expression after 24 h treatment of CpG SNAs or linear CpG ODNs. Mean fluorescence intensity (MFI) of (b) CD80 and (c) CD86 expression. (d) Percentage of CD80+/CD86+ double-positive cell population after treatment.

FIG. 16 shows representative flow cytometry data for CD80 and CD86 expression after a 24 hour treatment with CpG SNAs or linear CpG ODNs. Median value from triplicated experiment is shown.

FIG. 17 shows a CryoTEM image of LSNAs functionalized with 300 equivalents of class A CpG ODN. Scale bar: 100 nm.

FIG. 18 shows a CryoTEM image of LSNAs functionalized with 10,000 equivalents of class A CpG ODN. Scale bar: 100 nm.

FIG. 19 shows DLS characterization of liposomes and LSNAs.

FIG. 20 shows a CryoTEM image of mixed-CpG SNAs packed in two layers. Scale bars: 100 nm. (b) shows a zoomed in-view of the boxed portion with false color to show the layered structure.

FIG. 21 shows that dual-CpG SNAs promoted significant enhancement in dendritic cell activation (quantified by co-stimulation marker CD80 expression) in all dendritic cell subtypes collected from human peripheral blood mononuclear cells (PBMCs), compared to SNAs containing only CpG-B.

### DETAILED DESCRIPTION

Spherical nucleic acids (SNAs) are a novel class of nanoscale nucleic acids with demonstrated utility in a wide variety of biomedical applications, including gene regulation, cancer immunotherapy, and intracellular detection. To date, SNAs have been primarily generated from a single class of oligonucleotide, but the delivery of multiple classes of oligonucleotides with defined ratios to the same target cell would be advantageous for myriad biological applications. There are numerous applications where it may be beneficial to deliver multiple oligonucleotide sequences to the same target cell.¹⁶ Therefore, the present disclosure elucidates the additive effects of sequence multiplicity (i.e., multiple distinct oligonucleotide sequences on a single SNA construct) on the chemical and physical properties of SNAs in the context of a cell-regulatory process, immunostimulation.

Oligonucleotides have been commonly encapsulated inside liposomes or polymers, but such process is somewhat limited. Multiple sequence encapsulation efficiency is not guaranteed, as some sequences are more easily encapsulated than others, and the encapsulation ratio may have large variations since the encapsulated entities cannot exchange. Some sequences, such as class A CpG, cannot be encapsulated. Spherical nucleic acids can overcome these challenges and incorporate a variety of physically (e.g., structural features) and chemically (e.g., backbone chemistry) distinct oligonucleotide sequences with more controlled ratios. Sequence multiplicity spherical nucleic acids (SNAs) as described herein provide a platform for co-delivery of multiple oligonucleotide sequences at controlled ratios for synergistic therapeutic effects.

As used in this specification and the appended claims, the singular forms “a,” “an,” and “the” include plural reference unless the context clearly dictates otherwise.

The terms “class A CpG oligonucleotide” and “CpG-A” are interchangeable as used herein. The terms “class B CpG oligonucleotide” and “CpG-B” are interchangeable as used herein. The terms “class C CpG oligonucleotide” and “CpG-C” are interchangeable as used herein.

The terms “polynucleotide” and “oligonucleotide” are interchangeable as used herein.

As used herein, the term “about,” when used to modify a particular value or range, generally means within 20 percent, e.g., within 10 percent, 5 percent, 4 percent, 3 percent, 2 percent, or 1 percent of the stated value or range.

Unless otherwise stated, all ranges contemplated herein include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

### Spherical Nucleic Acids (SNAs)

As described herein, SNAs comprise a nanoparticle core surrounded by a shell of highly oriented oligonucleotides. In various embodiments, an oligonucleotide shell is formed when at least 10% of the available surface area of the exterior surface of a nanoparticle core includes an oligonucleotide. In further embodiments at least 20%, 30%, 40%, 50%, 60%, 70%, 80%, 90%, 95%, 96%, 97%, 98% or 99% of the available surface area of the exterior surface of the nanoparticle core includes an oligonucleotide. The oligonucleotides of the oligonucleotide shell may be oriented in a variety of directions. In some embodiments the oligonucleotides are oriented radially outwards. The oligonucleotide shell comprises one or more oligonucleotides attached to the external surface of the nanoparticle core. The nanoparticle core can either be organic (e.g., a liposome), inorganic (e.g., gold, silver, or platinum), or hollow (e.g., silica-based). The spherical architecture of the polynucleotide shell confers unique advantages over traditional nucleic acid delivery methods, including entry into nearly all cells independent of transfection agents and resistance to nuclease degradation. Furthermore, SNAs can penetrate biological barriers, including the blood-brain (see, e.g., U.S. Patent Application Publication No. 2015/0031745, incorporated by reference herein in its entirety) and blood-tumor barriers as well as the epidermis (see, e.g., U.S. Patent Application Publication No. 2010/0233270, incorporated by reference herein in its entirety).

SNAs can range in size from about 1 nanometer (nm) to about 500 nm, about 1 nm to about 400 nm, about 1 nm to about 300 nm, about 1 nm to about 200 nm, about 1 nm to about 150 nm, about 1 nm to about 100 nm, about 1 nm to about 90 nm, about 1 nm to about 80 nm in diameter, about 1 nm to about 70 nm in diameter, about 1 nm to about 60 nm in diameter, about 1 nm to about 50 nm in diameter, about 1 nm to about 40 nm in diameter, about 1 nm to about 30 nm in diameter, about 1 nm to about 20 nm in diameter, about 1 nm to about 10 nm, about 10 nm to about 150 nm in diameter, about 10 nm to about 140 nm in diameter, about 10 nm to about 130 nm in diameter, about 10 nm to about 120 nm in diameter, about 10 nm to about 110 nm in diameter, about 10 nm to about 100 nm in diameter, about 10 nm to about 90 nm in diameter, about 10 nm to about 80 nm in diameter, about 10 nm to about 70 nm in diameter, about 10 nm to about 60 nm in diameter, about 10 nm to about 50 nm in diameter, about 10 nm to about 40 nm in diameter, about 10 nm to about 30 nm in diameter, or about 10 nm to about 20 nm in diameter. In some embodiments, the SNA is about 1.4 nm in diameter. In further embodiments, the SNA is, is at least, or is less than about 100 nm, 90 nm, 80 nm, 70 nm, 60 nm, 50 nm, 40 nm, 30 nm, 20, or 10 nm in diameter (or in mean diameter when there are a plurality of SNAs). In further aspects, the disclosure provides a plurality of SNAs, each SNA comprising an oligonucleotide shell attached to the external surface of the nanoparticle core, wherein the oligonucleotide shell comprises or consists of a mixture of class A CpG oligonucleotides and class B CpG oligonucleotides. Thus, in some embodiments, the size of the plurality of SNAs is from about 10 nm to about 150 nm (mean diameter), about 10 nm to about 140 nm in mean diameter, about 10 nm to about 130 nm in mean diameter, about 10 nm to about 120 nm in mean diameter, about 10 nm to about 110 nm in mean diameter, about 10 nm to about 100 nm in mean diameter, about 10 nm to about 90 nm in mean diameter, about 10 nm to about 80 nm in mean diameter, about 10 nm to about 70 nm in mean diameter, about 10 nm to about 60 nm in mean diameter, about 10 nm to about 50 nm in mean diameter, about 10 nm to about 40 nm in mean diameter, about 10 nm to about 30 nm in mean diameter, or about 10 nm to about 20 nm in mean diameter. In some embodiments, the mean diameter of the plurality of SNAs is about 1.4 nm. In some embodiments, the diameter (or mean diameter for a plurality of SNAs) of the SNAs is from about 10 nm to about 150 nm, from about 30 to about 100 nm, or from about 40 to about 80 nm. In some embodiments, the size of the nanoparticles used in a method varies as required by their particular use or application. The variation of size is advantageously used to optimize certain physical characteristics of the SNAs, for example, the amount of surface area to which oligonucleotides may be attached as described herein. It will be understood that the foregoing diameters of SNAs can apply to the diameter of the nanoparticle core itself or to the diameter of the nanoparticle core and the oligonucleotide shell attached thereto. Further description of nanoparticle cores is provided herein below.

### Oligonucleotides

### CpG Oligonucleotides

Previous work has shown that SNAs with excellent immunostimulatory and immunomodulatory properties can be generated by employing synthetic oligonucleotides (e.g., oligodeoxynucleotides) that contain unmethylated cytosine-guanosine sequences, known as a CpG motif (CpG oligonucleotide (e.g., a CpG ODN), as the oligonucleotide shell.⁵⁻⁸ Treatment with immunostimulatory SNAs has demonstrated superior antitumor efficacy in multiple mouse models of cancer.^{5,8} CpG oligonucleotides (e.g., ODNs), oligonucleotides that mimic the genome of most bacteria and DNA viruses, are toll-like receptor 9 (TLR9) ligands that induce downstream activation of the myeloid differentiation primary response gene 88 (MyD88)-dependent signaling pathway, which leads to secretion of type I interferons (IFN) and cytokines.¹⁷⁻¹⁸ CpG oligonucleotides (e.g., ODNs) are generally categorized into a few classes. Among them, class A CpG oligonucleotides and class B CpG oligonucleotides were the first two classes identified and are optimal in activating different immune pathways, while other CpG classes show intermediate responses.¹⁹ In some embodiments, CpG-A has a partial phosphodiester (PO) backbone that comprises or consists of an internal CpG-containing palindrome and a partial phosphorothioate (PS) backbone that comprises or consists of poly(G) sequences on the terminals of the strands, while in further embodiments CpG-B comprises a fully PS backbone with hexamer CpG motifs. It has been reported in the literature that the combination of CpG-A and CpG-B ODNs have shown striking synergy, resulting in in vivo mouse survival after tumor challenge.²⁰

In various aspects, the present disclosure describes immune activation as a function of, for example and without limitation, the combination (and in some embodiments, the ratio) of class A CpG oligonucleotides and class B CpG oligonucleotides in the context of the SNA platform. Such dual-CpG SNAs are shown herein to enhance immune cell activation relative to a SNA comprising only a single class of CpG oligonucleotide (e.g., class B CpG oligonucleotides). In any of the aspects or embodiments of the disclosure, the SNA does not comprise a class C CpG oligonucleotide (CpG-C).

Class A CpG oligonucleotides, class B CpG oligonucleotides, and class C CpG oligonucleotides are generally understood in the art. CpG oligonucleotides are also described in, e.g., U.S. Pat. No. 6,552,006, Krieg, J Clin Invest. 117(5): 1184-1194 (2007), and Hanagata, Int J Nanomedicine 7:2181-2195 (2012), each of which is incorporated by reference herein in its entirety.

Class A CpG oligonucleotides are characterized by the ability to induce high levels of interferon-alpha while having minimal effects on TLR9-dependent NF-κB signaling and pro-inflammatory cytokine (e.g., IL-6) production. In general, class A CpG oligonucleotides are characterized by a central palindromic phosphodiester (PO) CpG motif and a poly(G) sequence at its 3′ end. Typically, at least the C of the CpG dinucleotide is unmethylated. In various embodiments, a class A CpG oligonucleotide comprises a central CpG-containing palindromic sequence and an at least partially phosphorothioated (PS) poly(G) sequence at its 5′ and/or 3′ end. In various embodiments, “at least partially phosphorothioated” means that about 40%, about 50%, about 60%, about 70%, about 80%, about 90%, or 100% of the guanines in the poly(G) sequence are phosphorothioated. In various embodiments, a class A CpG oligonucleotide contains all (i.e., 100%) PS or all PO internucleotide linkages. In some embodiments, the central palindromic CpG motif is or comprises TCAACGTTGA (SEQ ID NO: 1). In any of the aspects or embodiments of the disclosure, a class A CpG oligonucleotide is a DNA molecule. In any of the aspects or embodiments of the disclosure a class A CpG oligonucleotide is a single-stranded DNA molecule. In some embodiments, a class A CpG oligonucleotides comprises (i) an internal palindrome sequence containing a CpG motif and (ii) an at least partially phosphorothioated poly(G) sequence at its 5′ and/or 3′ ends.

Typically, CpG-B oligonucleotides comprise a full PS backbone with one or more CpG dinucleotides. In various embodiments, a CpG oligonucleotide of the disclosure comprises about 3 to 8 CpG motifs. In further embodiments, a CpG oligonucleotide of the disclosure comprises about 2 to 10, 2 to 9, 2 to 8, 2 to 7, 2 to 6, 2 to 5, 3 to 10, 3 to 9, 3 to 8, 3 to 7, or 3 to 6 CpG motifs. In further embodiments, a CpG oligonucleotide of the disclosure comprises or consists of 2, 3, 4, 5, 6, 7, 8, 9, 10, or more CpG motifs. CpG-B oligonucleotides strongly activate B cells and TLR9-dependent NF-κB signaling but weakly stimulate IFN-α secretion. In any of the aspects or embodiments of the disclosure, a class B CpG oligonucleotide comprises a fully phosphorothioated backbone (i.e., 100% phosphorothioate internucleotide linkages) with one or more CpG motifs. In some embodiments, the CpG motif is a hexamer CpG motif. In some embodiments, the hexamer CpG motif is or comprises GTCGTT. In any of the aspects or embodiments of the disclosure, a class B CpG oligonucleotide is a DNA molecule. In any of the aspects or embodiments of the disclosure a class B CpG oligonucleotide is a single-stranded DNA molecule.

In general, CpG-C oligonucleotides combine features of both classes A and B. Typically, CpG-C oligonucleotides comprise a complete PS backbone and a CpG-containing palindromic motif. C-Class CpG ODNs induce strong IFN-α production as well as B cell stimulation. In various embodiments, a class C CpG oligonucleotide comprises a phosphorothioate backbone, a phosphodiester backbone, or a combination thereof. In some embodiments, a class C CpG oligonucleotide is a DNA molecule. In some embodiments, a class C CpG oligonucleotide is a single-stranded DNA molecule.

As used herein, a “poly(G) sequence” is a nucleotide sequence of three or more consecutive G′s, e.g., 5′-GGG-3′, occurring at the 5′ end and/or the 3′ end of an oligonucleotide. As used herein, “CpG” refers to a “CpG-motif containing oligonucleotide,” i.e., the entire oligonucleotide. A “CpG motif” as used herein is the “CG” sequence within an oligonucleotide that is the ligand for TLR9 binding and activation (while the rest of the oligonucleotide sequence, in various embodiments, helps the oligonucleotide form a structure that facilitates CpG motif binding to TLR9. A “palindrome” or “palindromic sequence” refers to a sequence of nucleotides along an oligonucleotide strand that reads the same in the 5′→3′ direction as its complementary strand reads in the 5′→3′ direction. Palindromic sequences in a single stranded oligonucleotide can fold into a hairpin structure. By way of example, a palindromic sequence is TCAACGTTGA (SEQ ID NO: 1).

### Oligonucleotide Features

It will be understood that all features of oligonucleotides described herein (e.g., type (DNA/RNA/chimeric), single/double stranded, length, sequence, modified forms) apply to all oligonucleotides described herein, including CpG oligonucleotides and oligonucleotides that are not CpG oligonucleotides (e.g., an inhibitory oligonucleotide). The disclosure provides spherical nucleic acids (SNAs) comprising a nanoparticle core and an oligonucleotide shell attached to the external surface of the nanoparticle core. In some embodiments, the oligonucleotide shell comprises a mixture of class A CpG oligonucleotides and class B CpG oligonucleotides, wherein the SNA does not comprise a class C CpG oligonucleotide. In some embodiments, the oligonucleotide shell consists of a mixture of class A CpG oligonucleotides and class B CpG oligonucleotides. In some embodiments, the oligonucleotide shell comprises a mixture of class A CpG oligonucleotides and class B CpG oligonucleotides, wherein the ratio of class A CpG oligonucleotides to class B CpG oligonucleotides in the mixture is about 4:1 to about 1:1.

Oligonucleotides (e.g., CpG oligonucleotides) of the disclosure include, in various embodiments, DNA oligonucleotides, RNA oligonucleotides, modified forms thereof, or a combination thereof. Thus, in some embodiments, the CpG oligonucleotide is DNA oligonucleotide, RNA oligonucleotide, a modified oligonucleotide, or a combination thereof. In various embodiments, an oligonucleotide shell of the disclosure comprises DNA oligonucleotides, RNA oligonucleotides, modified oligonucleotides, or a combination thereof.

In any aspects or embodiments described herein, an oligonucleotide is single-stranded, double-stranded, or partially double-stranded. In various embodiments, an oligonucleotide shell of the disclosure comprises single stranded oligonucleotides, double stranded oligonucleotides, partially double stranded oligonucleotides, or a combination thereof.

As described herein, modified forms of oligonucleotides are also contemplated by the disclosure which include those having at least one modified internucleotide linkage. In some embodiments, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. The oligonucleotide incorporated with the universal base analogues is able to function, e.g., as a probe in hybridization. Examples of universal bases include but are not limited to 5′-nitroindole-2′-deoxyriboside, 3-nitropyrrole, inosine and hypoxanthine.

The term “nucleotide” or its plural as used herein is interchangeable with modified forms as discussed herein and otherwise known in the art. The term “nucleobase” or its plural as used herein is interchangeable with modified forms as discussed herein and otherwise known in the art. Nucleotides or nucleobases comprise the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30:613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, oligonucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide”.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In still further embodiments, oligonucleotide mimetics wherein both one or more sugar and/or one or more internucleotide linkage of the nucleotide units are replaced with “non-naturally occurring” groups. The bases of the oligonucleotide are maintained for hybridization. In some aspects, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

In still further embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligonucleotide consists of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═ (including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH=N—O—, —CH₂—NR^{H}—O—, —CH₂—O—N═ (including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}, —O—CH₂—S—, —S—CH₂—O—, —CH₂—CH₂—S—, —O—CH₂—CH₂—S—, —S—CH₂—CH═ (including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—; —O—S(O)₂—CH₂—, —O—P(O)₂—O—, —O—P(O,S)—O—, —O—P(S)₂—O—, —S—P(O)₂—O—, —S—P (O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(OCH₂CH₃)—O—, —O—PO(OCH₂CH₂S—R)—O—, —O—PO(BH₃)—O—, —O—PO(NHR^{N})—O—, —O—P(O)₂—NR^{H}H—, —NR^{H}—P(O)₂—O—, —O—P(O,NR^{H})—O—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—O—P(—O,S)—O—, —O—P(S)₂—O—, —NR^{H}P(O)₂—O—, —O—P(O,NR^{H})—O—, —O—PO(R″)—O—, —O—PO(CH₃)—O—, and —O—PO(NHR^{N})—O—, where R^{H} is selected form hydrogen and C₁₋₄-alkyl, and R″ is selected from C₁₋₆-alkyl and phenyl, are contemplated. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443.

Still other modified forms of oligonucleotides are described in detail in U.S. patent application No. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, or an RNA cleaving group. In one aspect, a modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl (2′-CH₂—CH═CH₂), 2′-O—allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In some aspects, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2 (3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O—methoxyethyl sugar modifications. See, U.S. Pat. No. 3,687,808, U.S. Pat. Nos. 4,845,205; 5, 130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In various aspects, an oligonucleotide of the disclosure (e.g., a CpG oligonucleotide), or a modified form thereof, is generally about 10 nucleotides to about 100 nucleotides in length. More specifically, an oligonucleotide of the disclosure is about 10 to about 90 nucleotides in length, about 10 to about 80 nucleotides in length, about 10 to about 70 nucleotides in length, about 10 to about 60 nucleotides in length, about 10 to about 50 nucleotides in length about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, about 18 to about 28 nucleotides in length, about 15 to about 26 nucleotides in length, and all oligonucleotides intermediate in length of the sizes specifically disclosed to the extent that the oligonucleotide is able to achieve the desired result. In further embodiments, an oligonucleotide of the disclosure is about 5 nucleotides to about 1000 nucleotides in length. In further embodiments, an oligonucleotide of the disclosure is about 5 to about 900 nucleotides in length, about 5 to about 800 nucleotides in length, about 5 to about 700 nucleotides in length, about 5 to about 600 nucleotides in length, about 5 to about 500 nucleotides in length about 5 to about 450 nucleotides in length, about 5 to about 400 nucleotides in length, about 5 to about 350 nucleotides in length, about 5 to about 300 nucleotides in length, about 5 to about 250 nucleotides in length, about 5 to about 200 nucleotides in length, about 5 to about 150 nucleotides in length, about 5 to about 100 nucleotides in length, about 5 to about 90 nucleotides in length, about 5 to about 80 nucleotides in length, about 5 to about 70 nucleotides in length, about 5 to about 60 nucleotides in length, about 5 to about 50 nucleotides in length, about 5 to about 40 nucleotides in length, about 5 to about 30 nucleotides in length, about 5 to about 20 nucleotides in length, about 5 to about 10 nucleotides in length, and all oligonucleotides intermediate in length of the sizes specifically disclosed to the extent that the oligonucleotide is able to achieve the desired result.

Accordingly, in various embodiments, an oligonucleotide of the disclosure is or is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or more nucleotides in length. In further embodiments, an oligonucleotide of the disclosure is less than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or more nucleotides in length. In various embodiments, a SNA comprises a plurality of oligonucleotides that all have the same length/sequence, while in some embodiments, the plurality of oligonucleotides comprises one or more oligonucleotide that have a different length and/or sequence relative to at least one other oligonucleotide in the plurality.

In some embodiments, the oligonucleotide is an aptamer. Accordingly, all features and aspects of oligonucleotides described herein (e.g., length, type (DNA, RNA, modified forms thereof), optional presence of spacer) also apply to aptamers. Aptamers are oligonucleotide sequences that can be evolved to bind to various target analytes of interest. Aptamers may be single stranded, double stranded, or partially double stranded.

Methods of attaching detectable markers (e.g., fluorophores, radiolabels) and additional moieties (e.g., an antibody) as described herein to an oligonucleotide are known in the art.

In various aspects, a SNA of the disclosure has the ability to bind to a plurality of targets (e.g., polynucleotides, proteins). In some embodiments, a SNA further comprises one or more oligonucleotides that are not CpG oligonucleotides. Such oligonucleotides that are not CpG oligonucleotides may be inhibitory oligonucleotides as described herein. Thus, in some embodiments, a SNA of the disclosure comprises one or more oligonucleotides having a sequence sufficiently complementary to the target polynucleotide to hybridize under the conditions being used. In some embodiments, the SNA comprises two or more oligonucleotides that are not identical, i.e., at least one of the attached oligonucleotides differ from at least one other attached oligonucleotide in that it has a different length and/or a different sequence. For example, if a specific polynucleotide is targeted, a single SNA has the ability to bind to multiple copies of the same target. In some embodiments, a single SNA has the ability to bind to different targets Accordingly, in various aspects, a single SNA may be used in a method to inhibit expression of more than one gene product. In various embodiments, oligonucleotides are thus used to target specific polynucleotides, whether at one or more specific regions in the target polynucleotide, or over the entire length of the target polynucleotide as the need may be to effect a desired level of inhibition of gene expression. Accordingly, in various aspects and embodiments of the disclosure, a SNA of the disclosure possesses immunostimulatory activity and inhibition of gene expression activity.

Nanoparticle surface density. A surface density adequate to make the nanoparticles stable and the conditions necessary to obtain it for a desired combination of nanoparticles and oligonucleotides can be determined empirically. In general, oligonucleotides of the disclosure are attached to the nanoparticle core at a surface density of at least about 2 pmoles/cm². In some aspects, the surface density is about or at least about 15 pmoles/cm². Methods are also provided wherein oligonucleotides are bound to the nanoparticle core at a surface density of at least 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm², at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more. Alternatively, the density of oligonucleotides attached to the SNA is measured by the number of oligonucleotides attached to the SNA. With respect to the surface density of oligonucleotides attached to an SNA, it is contemplated that a SNA as described herein comprises about 1 to about 2,500, or about 1 to about 500 oligonucleotides on its surface. In various embodiments, a SNA comprises about 10 to about 500, or about 10 to about 300, or about 10 to about 200, or about 10 to about 190, or about 10 to about 180, or about 10 to about 170, or about 10 to about 160, or about 10 to about 150, or about 10 to about 140, or about 10 to about 130, or about 10 to about 120, or about 10 to about 110, or about 10 to about 100, or 10 to about 90, or about 10 to about 80, or about 10 to about 70, or about 10 to about 60, or about 10 to about 50, or about 10 to about 40, or about 10 to about 30, or about 10 to about 20 oligonucleotides in the shell of oligonucleotides attached to the nanoparticle core. In some embodiments, a SNA comprises about 80 to about 140 oligonucleotides in the shell of oligonucleotides attached to the nanoparticle core. In further embodiments, a SNA comprises at least about 5, 10, 20, 30, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 oligonucleotides in the shell of oligonucleotides attached to the nanoparticle core. In further embodiments, a SNA comprises or consists of 1, 2, 3, 4, 5, 10, 20, 30, 40, 45, 50, 55, 60, 65, 70, 75, 80, 85, 90, 95, 100, 105, 110, 115, 120, 125, 127, 130, 135, 140, 145, 150, 155, 160, 165, 170, 175, 180, 185, 190, 195, 200, 300, 400, 500, 600, 700, 800, 900, or 1000 oligonucleotides in the shell of oligonucleotides attached to the nanoparticle core. In still further embodiments, the shell of oligonucleotides attached to the nanoparticle core of the SNA comprises 2, 3, 4, 5, 6, 7, 8, 9, 10, 11, 12, 13, 14, 15, 16, 17, 18, 19, 20, or more oligonucleotides. In some embodiments, the shell of oligonucleotides attached to the nanoparticle core of the SNA comprises at least 20 oligonucleotides. In some embodiments, the shell of oligonucleotides attached to the nanoparticle core of the SNA consists of 127.5 oligonucleotides. In some embodiments, the shell of oligonucleotides attached to the nanoparticle core of the SNA comprises or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more oligonucleotides. In various embodiments, about 2 to about 1000, or about 2 to about 100 oligonucleotides are attached to the external surface of a nanoparticle core. In further embodiments, about 10 to about 1000, or about 10 to about 750, or about 10 to about 500, or about 10 to about 250, or about 10 to about 100, or about 50 to about 1000, or about 50 to about 750, or about 50 to about 500, or about 50 to about 250, or about 100 to about 1000, or about 100 to about 500, or about 2 to about 90, or about 2 to about 80, or about 2 to about 70, or about 2 to about 60, or about 2 to about 50, or about 2 to about 40, or about 2 to about 30, or about 2 to about 20, or about 2 to about 10, or about 10 to about 100, or about 10 to about 90, or about 10 to about 80, or about 10 to about 70, or about 10 to about 60, or about 10 to about 50, or about 10 to about 40, or about 10 to about 30, or about 10 to about 20, or about 20 to about 100, or about 20 to about 90, or about 20 to about 80, or about 20 to about 70, or about 20 to about 60, or about 20 to about 50, or about 20 to about 40, or about 20 to about 30 oligonucleotides are attached to the external surface of a nanoparticle core. In still further embodiments, at least 1, at least 2, at least 3, at least 4, at least 5, at least 6, at least 7, at least 8, at least 9, or at least 10 oligonucleotides are attached to the nanoparticle core.

In any of the aspects or embodiments of the disclosure, a SNA comprises a mixture of class A CpG oligonucleotides and class B CpG oligonucleotides. In various embodiments, the number of class A CpG oligonucleotides and class B CpG oligonucleotides attached to a nanoparticle core are varied to achieve SNAs having various ratios of class A CpG oligonucleotides to class B CpG oligonucleotides. In some embodiments, the ratio of class A CpG oligonucleotides to class B CpG oligonucleotides in the mixture is or is about 4:1 to about 1:1. In some embodiments, the ratio of class A CpG oligonucleotides to class B CpG oligonucleotides in the mixture is or is about 7:3. In further embodiments, the ratio of class A CpG oligonucleotides to class B CpG oligonucleotides in the mixture is or is about 4:1 to about 2:1, or about 4:1 to about 3:1, or about 3:1 to about 2:1, or about 3:1 to about 1:1. In still further embodiments, the ratio of class A CpG oligonucleotides to class B CpG oligonucleotides in the mixture is, is about, or is at least about 1:1, 1:9, 1:4, 3:7, 9:1, 2:1, 7:3, 6:4, 3:1, or 4:1. In still further embodiments, the ratio of class A CpG oligonucleotides to class B CpG oligonucleotides in the mixture is less than about 1:1, 1:9, 1:4, 3:7, 9:1, 2:1, 7:3, 6:4, 3:1, or 4:1.

In some embodiments, a SNA of the disclosure further comprises one or more class C CpG oligonucleotides.

Spacers. In some aspects, an oligonucleotide (e.g., CpG oligonucleotide) is attached to a nanoparticle through a spacer (and, in some embodiments, additionally through a linker). “Spacer” as used herein means a moiety that serves to increase distance between the nanoparticle and the oligonucleotide, or to increase distance between individual oligonucleotides when attached to the nanoparticle in multiple copies. Thus, spacers are contemplated being located between individual oligonucleotides in tandem, whether the oligonucleotides have the same sequence or have different sequences.

In some aspects, the spacer when present is an organic moiety. In some aspects, the spacer is a polymer, including but not limited to a water-soluble polymer, a nucleic acid, a polypeptide, an oligosaccharide, a carbohydrate, a lipid, an ethylglycol, or a combination thereof. In any of the aspects or embodiments of the disclosure, the spacer is an oligo (ethylene glycol)-based spacer. In various embodiments, an oligonucleotide comprises 1, 2, 3, 4, 5, or more spacer (e.g., Spacer-18 (hexaethyleneglycol)) moieties. In further embodiments, the spacer is an alkane-based spacer (e.g., C12). In some embodiments, the spacer is an oligonucleotide spacer (e.g., T5). An oligonucleotide spacer may have any sequence that does not interfere with the ability of the oligonucleotide to perform an intended function (e.g., stimulate an immune response or inhibit gene expression). In certain aspects, the bases of the oligonucleotide spacer are all adenylic acids, all thymidylic acids, all cytidylic acids, all guanylic acids, all uridylic acids, or all some other modified base.

In various embodiments, the length of the spacer is or is equivalent to at least about 2 nucleotides, at least about 3 nucleotides, at least about 4 nucleotides, at least about 5 nucleotides, 5-10 nucleotides, 10 nucleotides, 20 nucleotides, 10-30 nucleotides, or even greater than 30 nucleotides.

Oligonucleotide attachment to a nanoparticle core. Oligonucleotides contemplated for use according to the disclosure include those attached to a nanoparticle core through any means (e.g., covalent or non-covalent attachment). Regardless of the means by which the oligonucleotide is attached to the nanoparticle, attachment in various aspects is effected through a 5′ linkage, a 3′ linkage, some type of internal linkage, or any combination of these attachments. In some embodiments, the oligonucleotide is covalently attached to a nanoparticle. In further embodiments, the oligonucleotide is non-covalently attached to a nanoparticle. In further embodiments, the oligonucleotide is attached to a nanoparticle via a combination of covalent and non-covalent linkage.

Methods of attachment are known to those of ordinary skill in the art and are described in U.S. Publication No. 2009/0209629, which is incorporated by reference herein in its entirety. Methods of attaching RNA to a nanoparticle are generally described in PCT/US2009/65822, which is incorporated by reference herein in its entirety. Methods of associating oligonucleotides with a liposomal particle are described in PCT/US2014/068429, which is incorporated by reference herein in its entirety.

Methods of attaching oligonucleotides to a protein core are described, e.g., in U.S. Patent Application Publication No. 2017/0232109 and Brodin et al., J Am Chem Soc. 137 (47): 14838-41 (2015), each of which is incorporated by reference herein in its entirety. In general, a polynucleotide can be modified at a terminus with an alkyne moiety, e.g., a DBCO—type moiety for reaction with the azide of the protein surface:

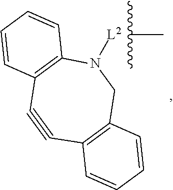

where L is a linker to a terminus of the polynucleotide. L² can be C₁₋₁₀ alkylene, —C(O)—C₁₋₁₀ alkylene-Y—, and —C(O)—C₁₋₁₀ alkylene-Y—C₁₋₁₀ alkylene-(OCH₂CH₂)ₘ—Y—; wherein each Y is independently selected from the group consisting of a bond, C(O), O, NH, C(O)NH, and NHC(O); and m is 0, 1, 2, 3, 4, or 5. For example, the DBCO functional group can be attached via a linker having a structure of

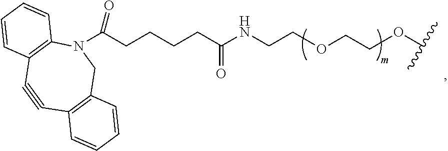

where the terminal “O” is from a terminal nucleotide on the polynucleotide. Use of this DBCO—type moiety results in a structure between the polynucleotide and the protein, in cases where a surface amine is modified, of:

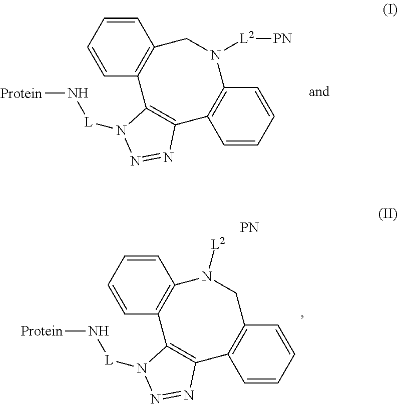

where L and L² are each independently selected from C₁₋₁₀ alkylene, —C(O)—C₁₋₁₀ alkylene-Y—, and —C(O)—C₁₋₁₀ alkylene-Y—C₁₋₁₀ alkylene-(OCH₂CH₂)ₘ—Y—; each Y is independently selected from the group consisting of a bond, C(O), O, NH, C(O)NH, and NHC(O); m is 0, 1, 2, 3, 4, or 5; and PN is the polynucleotide. Similar structures where a surface thiol or surface carboxylate of the protein are modified can be made in a similar fashion to result in comparable linkage structures.

The protein can be modified at a surface functional group (e.g., a surface amine, a surface carboxylate, a surface thiol) with a linker that terminates with an azide functional group: Protein-X-L-N₃, X is from a surface amino group (e.g., —NH—), carboxylic group (e.g., —C(O)— or —C(O)O—), or thiol group (e.g., —S—) on the protein; L is selected from C₁₋₁₀ alkylene, —Y—C(O)—C₁₋₁₀ alkylene-Y—, and —Y—C(O)—C₁₋₁₀ alkylene-Y—C₁₋₁₀ alkylene-(OCH₂CH₂)ₘ—Y—; each Y is independently selected from the group consisting of a bond, C(O), O, NH, C(O)NH, and NHC(O); and m is 0, 1, 2, 3, 4, or 5. Introduction of the “L-N₃” functional group to the surface moiety of the protein can be accomplished using well-known techniques. For example, a surface amine of the protein can be reacted with an activated ester of a linker having a terminal N₃ to form an amide bond between the amine of the protein and the carboxylate of the activated ester of the linker reagent.

The polynucleotide can be modified to include an alkyne functional group at a terminus of the polynucleotide: Polynucleotide-L₂-X-≡-R; L² is selected from C₁₋₁₀ alkylene, —C(O)—C₁₋₁₀ alkylene-Y—, and —C(O)—C₁₋₁₀ alkylene-Y—C₁₋₁₀ alkylene-(OCH₂CH₂)ₘ—Y—; each Y is independently selected from the group consisting of a bond, C(O), O, NH, C(O)NH, and NHC(O); m is 0, 1, 2, 3, 4, or 5; and X is a bond and R is H or C₁₋₁₀alkyl; or X and R together with the carbons to which they are attached form a 8-10 membered carbocyclic or 8-10 membered heterocyclic group. In some cases, the polynucleotide has a structure

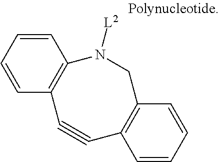

The protein, with the surface modified azide, and the polynucleotide, with a terminus modified to include an alkyne, can be reacted together to form a triazole ring in the presence of a copper (II) salt and a reducing agent to generate a copper (I) salt in situ. In some cases, a copper (I) salt is directly added. Contemplated reducing agents include ascorbic acid, an ascorbate salt, sodium borohydride, 2-mercaptoethanol, dithiothreitol (DTT), hydrazine, lithium aluminum hydride, diisobutylaluminum hydride, oxalic acid, Lindlar catalyst, a sulfite compound, a stannous compound, a ferrous compound, sodium amalgam, tris(2-carboxyethyl) phosphine, hydroquinone, and mixtures thereof.

The surface functional group of the protein can be attached to the polynucleotide using other attachment chemistries. For example, a surface amine can be directed conjugated to a carboxylate or activated ester at a terminus of the polynucleotide, to form an amide bond. A surface carboxylate can be conjugated to an amine on a terminus of the polynucleotide to form an amide bond. Alternatively, the surface carboxylate can be reacted with a diamine to form an amide bond at the surface carboxylate and an amine at the other terminus. This terminal amine can then be modified in a manner similar to that for a surface amine of the protein. A surface thiol can be conjugated with a thiol moiety on the polynucleotide to form a disulfide bond. Alternatively, the thiol can be conjugated with an activated ester on a terminus of a polynucleotide to form a thiocarboxylate.

### Nanoparticle Core

In general, nanoparticles contemplated by the disclosure include any compound or substance with a high loading capacity for an oligonucleotide as described herein, including for example and without limitation, a protein, a metal, a semiconductor, a liposomal particle, a polymer-based particle (e.g., a poly (lactic-co-glycolic acid) (PLGA) particle), insulator particle compositions, and a dendrimer (organic versus inorganic). Thus, in various embodiments, the nanoparticle core is organic (e.g., a liposome), inorganic (e.g., gold, silver, or platinum), porous (e.g., silica-based or metal organic-framework-based), or hollow. In any of the aspects or embodiments of the disclosure, the nanoparticle core is a protein core.

Thus, the disclosure contemplates nanoparticle cores that comprise a variety of inorganic materials including, but not limited to, metals, semi-conductor materials or ceramics as described in U.S. Patent Publication No 20030147966. For example, metal-based nanoparticles include those described herein. In various embodiments, the nanoparticle core is a metallic core, a semiconductor core, an insulator core, an upconverting core, a micellar core, a dendrimer core, a liposomal core, a polymer core, a metal-organic framework core, a protein core, or a combination thereof. Ceramic nanoparticle materials include, but are not limited to, brushite, tricalcium phosphate, alumina, silica, and zirconia. Organic materials from which nanoparticles are produced include carbon. Nanoparticle polymers include polystyrene, silicone rubber, polycarbonate, polyurethanes, polypropylenes, polymethylmethacrylate, polyvinyl chloride, polyesters, polyethers, and polyethylene. Biodegradable, biopolymer (e.g., polypeptides such as BSA, polysaccharides, etc.), other biological materials (e.g., carbohydrates), and/or polymeric compounds are also contemplated for use in producing nanoparticles. In some embodiments, the polymer is polylactide, a polylactide-polyglycolide copolymer, a polycaprolactone, a polyacrylate, alginate, albumin, polypyrrole, polythiophene, polyaniline, polyethylenimine, poly (methyl methacrylate), chitosan, or a related structure. In some embodiments, the polymer is poly (lactic-co-glycolic acid) (PLGA).

Liposomal particles, for example as disclosed in International Patent Application No. PCT/US2014/068429 and U.S. Pat. No. 10,792,251 (each of which is incorporated by reference herein in its entirety) are also contemplated by the disclosure. Hollow particles, for example as described in U.S. Patent Publication Number 2012/0282186 (incorporated by reference herein in its entirety) are also contemplated herein. Liposomes of the disclosure have at least a substantially spherical geometry, an internal side and an external side, and comprise a lipid bilayer. The lipid bilayer comprises, in various embodiments, a lipid from the phosphocholine family of lipids or the phosphoethanolamine family of lipids. In various embodiments, the lipid is 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), cardiolipin, lipid A, cell-derived lipids, or a combination thereof.

In some embodiments, the nanoparticle is metallic, and in various aspects, the nanoparticle is a colloidal metal. Thus, in various embodiments, nanoparticles useful in the practice of the methods include metal (including for example and without limitation, gold, silver, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials. Other nanoparticles useful in the practice of the invention include, also without limitation, ZnS, ZnO, Ti, TiO₂, Sn, SnO₂, Si, SiO₂, Fe, Fe₃O₄, Fe₂O₃, Ag, Cu, Ni, Al, steel, cobalt-chrome alloys, Cd, titanium alloys, AgI, AgBr, HgI₂, PbS, PbSe, ZnTe, CdTe, In₂S₃, In₂Se₃, Cd₃P₂, Cd₃As₂, InAs, and GaAs. Methods of making ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshavsky, et al., J. Am. Chem. Soc., 112, 9438 (1990); Ushida et al., J. Phys. Chem., 95, 5382 (1992). In some embodiments, the nanoparticle is an iron oxide nanoparticle. In further embodiments, the nanoparticle core is gold, silver, platinum, aluminum, palladium, copper, cobalt, indium, cadmium selenide, iron oxide, fullerene, metal-organic framework, zinc sulfide, or nickel.

Methods of making metal, semiconductor and magnetic nanoparticles are well-known in the art. See, for example, Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Preparation of polyalkylcyanoacrylate nanoparticles prepared is described in Fattal, et al., J. Controlled Release (1998) 53:137-143 and U.S. Pat. No. 4,489,055. Methods for making nanoparticles comprising poly(D-glucaramidoamine)s are described in Liu, et al., J. Am. Chem. Soc. (2004) 126:7422-7423. Preparation of nanoparticles comprising polymerized methylmethacrylate (MMA) is described in Tondelli, et al., Nucl. Acids Res. (1998) 26:5425-5431, and preparation of dendrimer nanoparticles is described in, for example Kukowska-Latallo, et al., Proc. Natl. Acad. Sci. USA (1996) 93:4897-4902 (Starburst polyamidoamine dendrimers).

Suitable nanoparticles are also commercially available from, for example, Ted Pella, Inc. (gold), Amersham Corporation (gold) and Nanoprobes, Inc. (gold).

In any of the aspects or embodiments of the disclosure, the nanoparticle core is a protein. As used herein, “protein” is used interchangeably with “polypeptide” and refers to one or more polymers of amino acid residues. In various embodiments of the disclosure, a protein core comprises or consists of a single protein (i.e., a single polymer of amino acids), a multimeric protein, a peptide (e.g., a polymer of amino acids that between about 2 and 50 amino acids in length), or a synthetic fusion protein of two or more proteins. Synthetic fusion proteins include, without limitation, an expressed fusion protein (expressed from a single gene) and post-expression fusions where proteins are conjugated together chemically. Protein/oligonucleotide core-shell nanoparticles are also generally described in U.S. Patent Application Publication No. 2017/0232109, which is incorporated by reference herein in its entirety.

Proteins are understood in the art and include without limitation an enzyme, a therapeutic protein (e.g., adenosine deaminase, phosphatase and tensin homolog (PTEN), an interleukin (e.g., IL-1, IL-2, IL-6, IL-10, IL-12, etc.), an interferon (e.g., IFN-α, IFN-β, IFN-γ), or tumor necrosis factor alpha (TNF-α), a structural protein (e.g., actin), an antibody, a storage protein (e.g., ovalbumin), a transport protein (e.g., hemoglobin), a hormone (e.g., insulin), a receptor protein (e.g., G-Protein Coupled Receptors), a motor protein (e.g., kinesin, dynein, or myosin), an immunogenic protein (e.g., ovalbumin or a stimulator of interferon genes (STING) protein) or a fluorescent protein (e.g., green fluorescent protein (GFP)). In various embodiments, proteins contemplated by the disclosure include without limitation those having catalytic, signaling, therapeutic, or transport activity.

Proteins of the present disclosure may be either naturally occurring or non-naturally occurring. Proteins optionally include a spacer as described herein.

Naturally occurring proteins include without limitation biologically active proteins (including antibodies) that exist in nature or can be produced in a form that is found in nature by, for example, chemical synthesis or recombinant expression techniques. Thus, a protein core of the disclosure is or comprises, in some embodiments, an antibody. Naturally occurring proteins also include lipoproteins and post-translationally modified proteins, such as, for example and without limitation, glycosylated proteins. Antibodies contemplated for use in the methods and compositions of the present disclosure include without limitation antibodies that recognize and associate with a target molecule either in vivo or in vitro.

Structural proteins contemplated by the disclosure include without limitation actin, tubulin, collagen, and elastin.

Non-naturally occurring proteins contemplated by the present disclosure include but are not limited to synthetic proteins, as well as fragments, analogs and variants of naturally occurring or non-naturally occurring proteins as defined herein. Non-naturally occurring proteins also include proteins or protein substances that have D-amino acids, modified, derivatized, or non-naturally occurring amino acids in the D-or L-configuration and/or peptidomimetic units as part of their structure. The term “peptide” typically refers to short (e.g., about 2-50 amino acids in length) polypeptides/proteins. Non-naturally occurring proteins are prepared, for example, using an automated protein synthesizer or, alternatively, using recombinant expression techniques using a modified polynucleotide which encodes the desired protein.

As used herein a “fragment” of a protein is meant to refer to any portion of a protein smaller than the full-length protein or protein expression product. As used herein an “analog” refers to any of two or more proteins substantially similar in structure and having the same biological activity, but can have varying degrees of activity, to either the entire molecule, or to a fragment thereof. Analogs differ in the composition of their amino acid sequences based on one or more mutations involving substitution, deletion, insertion and/or addition of one or more amino acids for other amino acids. Substitutions can be conservative or non-conservative based on the physico-chemical or functional relatedness of the amino acid that is being replaced and the amino acid replacing it. As used herein a “variant” refers to a protein or analog thereof that is modified to comprise additional chemical moieties not normally a part of the molecule. Such moieties may modulate, for example and without limitation, the molecule's solubility, absorption, and/or biological half-life. Moieties capable of mediating such effects are disclosed in Remington's Pharmaceutical Sciences (1980). Procedures for coupling such moieties to a molecule are well known in the art. In various aspects, proteins are modified by glycosylation, pegylation, and/or polysialylation.

Fusion proteins, including fusion proteins wherein one fusion component is a fragment or a mimetic, are also contemplated. A “mimetic” as used herein means a peptide or protein having a biological activity that is comparable to the protein of which it is a mimetic. By way of example, an endothelial growth factor mimetic is a peptide or protein that has a biological activity comparable to the native endothelial growth factor. The term further includes peptides or proteins that indirectly mimic the activity of a protein of interest, such as by potentiating the effects of the natural ligand of the protein of interest.

Proteins include antibodies along with fragments and derivatives thereof, including but not limited to Fab′ fragments, F(ab)2 fragments, Fv fragments, Fc fragments, one or more complementarity determining regions (CDR) fragments, individual heavy chains, individual light chain, dimeric heavy and light chains (as opposed to heterotetrameric heavy and light chains found in an intact antibody, single chain antibodies (scAb), humanized antibodies (as well as antibodies modified in the manner of humanized antibodies but with the resulting antibody more closely resembling an antibody in a non-human species), chelating recombinant antibodies (CRABs), bispecific antibodies and multispecific antibodies, and other antibody derivative or fragments known in the art.

### SNA Synthesis

As described herein, SNAs of the disclosure generally comprise a nanoparticle core and an oligonucleotide shell comprising a mixture of CpG oligonucleotides. CpG oligonucleotides of the disclosure are immunostimulatory oligonucleotides, that is, they are capable of activating an immune cell. SNAs of the disclosure are therefore synthesized such that an oligonucleotide shell comprising a mixture of CpG oligonucleotides is attached to the external surface of a nanoparticle core. In some embodiments, the disclosure contemplates that a SNA comprises one or more oligonucleotides encapsulated within the SNA (e.g., within the nanoparticle core of the SNA). Syntheses of SNAs is described in detail herein (e.g., Example 1).

### Uses of SNAs in Gene Regulation/Therapy

It is contemplated that in any of the aspects or embodiments of the disclosure, a SNA as disclosed herein possesses the ability to regulate gene expression. Thus, in some embodiments, a SNA of the disclosure comprises a nanoparticle core and an oligonucleotide shell attached to the external surface of the nanoparticle core, wherein the oligonucleotide shell comprises a mixture of class A CpG oligonucleotides and class B CpG oligonucleotides, and the SNA further comprises one or more oligonucleotides having gene regulatory activity (e.g., inhibition of target gene expression or target cell recognition). In some embodiments, a SNA of the disclosure comprises one or more oligonucleotides that is an inhibitory oligonucleotide as described herein. Accordingly, in some embodiments the disclosure provides methods for inhibiting gene product expression, and such methods include those wherein expression of a target gene product is inhibited by about or at least about 5%, about or at least about 10%, about or at least about 15%, about or at least about 20%, about or at least about 25%, about or at least about 30%, about or at least about 35%, about or at least about 40%, about or at least about 45%, about or at least about 50%, about or at least about 55%, about or at least about 60%, about or at least about 65%, about or at least about 70%, about or at least about 75%, about or at least about 80%, about or at least about 85%, about or at least about 90%, about or at least about 95%, about or at least about 96%, about or at least about 97%, about or at least about 98%, about or at least about 99%, or 100% compared to gene product expression in the absence of a SNA. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of SNA and a specific oligonucleotide. In various aspects, the methods include use of an oligonucleotide branch sufficiently complementary to a target polynucleotide as described herein.

Accordingly, methods of utilizing a SNA of the disclosure in gene regulation therapy are provided. This method comprises the step of hybridizing a target polynucleotide encoding the gene product with one or more oligonucleotides of a SNA that are complementary to all or a portion of the target polynucleotide, wherein hybridizing between the target polynucleotide and the oligonucleotide occurs over a length of the target polynucleotide with a degree of complementarity sufficient to inhibit expression of the gene product. The inhibition of gene expression may occur in vivo or in vitro.

In various embodiments, the inhibitory oligonucleotide utilized in the methods of the disclosure is RNA, DNA, or a modified form thereof. In various embodiments, the inhibitory oligonucleotide is antisense DNA, small interfering RNA (siRNA), an aptamer, a short hairpin RNA (shRNA), a DNAzyme, or an aptazyme.

### Uses of SNAs in Immune Regulation

Toll-like receptors (TLRs) are a class of proteins, expressed in sentinel cells, that play a key role in regulation of innate immune system. The mammalian immune system uses two general strategies to combat infectious diseases. Pathogen exposure rapidly triggers an innate immune response that is characterized by the production of immunostimulatory cytokines, chemokines and polyreactive IgM antibodies. The innate immune system is activated by exposure to Pathogen Associated Molecular Patterns (PAMPs) that are expressed by a diverse group of infectious microorganisms. The recognition of PAMPs is mediated by members of the Toll-like family of receptors. TLR receptors, such as TLR 4, TLR 8 and TLR 9 that respond to specific oligonucleotides are located inside special intracellular compartments, called endosomes. The mechanism of modulation of, for example and without limitation, TLR 4, TLR 8 and TLR 9 receptors, is based on DNA-protein interactions.

As described herein, synthetic immunostimulatory oligonucleotides that contain CpG motifs that are similar to those found in bacterial DNA stimulate a similar response of the TLR receptors. Thus, CpG oligonucleotides of the disclosure have the ability to function as TLR agonists. Other TLR agonists contemplated by the disclosure include, without limitation, single-stranded RNA and small molecules (e.g.,R848 (Resiquimod)). Therefore, immunomodulatory oligonucleotides have various potential therapeutic uses, including treatment of immune deficiency and cancer. Thus, in some embodiments, a SNA of the disclosure is used in a method to modulate the activity of a toll-like receptor (TLR).

In further embodiments, a SNA of the disclosure comprises an oligonucleotide that is a TLR antagonist. In some embodiments, the immunostimulatory oligonucleotide is a double-stranded DNA (dsDNA).

In some embodiments, down regulation of the immune system would involve knocking down the gene responsible for the expression of the Toll-like receptor. This antisense approach involves use of a SNA of the disclosure to knock down the expression of any toll-like protein.

Accordingly, in some embodiments, methods of utilizing SNAs as described herein for modulating toll-like receptors are disclosed. The method either up-regulates or down-regulates the Toll-like-receptor activity through the use of a TLR agonist or a TLR antagonist, respectively. The method comprises contacting a cell having a toll-like receptor with a SNA of the disclosure, thereby modulating the activity and/or the expression of the toll-like receptor. The toll-like receptors modulated include one or more of toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, and/or toll-like receptor 13.

### Uses of SNAs to Treat a Disorder

In some embodiments, a SNA of the disclosure is used to treat a disorder. As used herein, “treat” or “treating” means to eliminate, reduce, or ameliorating the disorder or one or more symptoms thereof. Thus, in some aspects, the disclosure provides methods of treating a disorder comprising administering an effective amount of the SNA of the disclosure to a subject (e.g., a human subject) in need thereof, wherein the administering treats the disorder. In various embodiments, the disorder is cancer, an infectious disease, an autoimmune disease, or a combination thereof. An “effective amount” of the SNA is an amount sufficient to activate an innate immune response. Thus, methods of activating an innate immune response are also contemplated herein, such methods comprising administering a SNA of the disclosure to a subject in need thereof in an amount effective to activate an innate immune response in the subject.

A SNA of the disclosure can be administered via any suitable route, such as parenteral administration, intramuscular injection, subcutaneous injection, intradermal administration, and/or mucosal administration such as oral or intranasal. Additional routes of administration include but are not limited to intravenous, intraperitoneal, intranasal administration, intra-vaginal, intra-rectal, and oral administration. A combination of different routes of administration, separately or at the same time, is also contemplated by the disclosure.

### Compositions

The disclosure also provides compositions that comprise a SNA of the disclosure, or a plurality thereof. In some embodiments, the composition further comprises a pharmaceutically acceptable carrier. The term “carrier” refers to a vehicle within which the SNA as described herein is administered to a subject. Any conventional media or agent that is compatible with the SNAs according to the disclosure can be used. The term carrier encompasses diluents, excipients, adjuvants and a combination thereof.

### Additional Agents

The SNAs provided herein optionally further comprise an additional agent, or a plurality thereof. The additional agent is, in various embodiments, simply associated with an oligonucleotide in the shell of oligonucleotides attached to the nanoparticle core of the SNA, and/or the additional agent is associated with the nanoparticle core of the SNA, and/or the additional agent is encapsulated in the SNA. In some embodiments, the additional agent is associated with the end of an oligonucleotide that is not attached to the nanoparticle core (e.g., if the oligonucleotide is attached to the nanoparticle core through its 3′ end, then the additional agent is associated with the 5′ end of the oligonucleotide). Alternatively, in some embodiments, the additional agent is associated with the end of an oligonucleotide that is attached to the nanoparticle core (e.g., if the oligonucleotide is attached to the nanoparticle core through its 3′ end, then the additional agent is associated with the 3′ end of the oligonucleotide). In some embodiments, the additional agent is covalently associated with an oligonucleotide in the shell of oligonucleotides that is attached to the nanoparticle core of the SNA. In some embodiments, the additional agent is non-covalently associated with an oligonucleotide in the shell of oligonucleotides that is attached to the nanoparticle core of the SNA. However, it is understood that the disclosure provides SNAs wherein one or more additional agents are both covalently and non-covalently associated with oligonucleotides in the shell of oligonucleotides that is attached to the nanoparticle core of the SNA. It will also be understood that non-covalent associations include hybridization, protein binding, and/or hydrophobic interactions. In some embodiments, an additional agent is administered separately from a SNA of the disclosure. Thus, in some embodiments, an additional agent is administered before, after, or concurrently with a SNA of the disclosure to treat a disorder.

Additional agents contemplated by the disclosure include without limitation a protein (e.g., a therapeutic protein), a small molecule, a peptide, or a combination thereof. These additional agents are described herein. Proteins and peptides are described herein and may be used as a nanoparticle core, an additional agent, or both. In some embodiments, the additional agent is a chemotherapeutic agent.

The term “small molecule,” as used herein, refers to a chemical compound or a drug, or any other low molecular weight organic compound, either natural or synthetic. By “low molecular weight” is meant compounds having a molecular weight of less than 1500 Daltons, typically between 100 and 700 Daltons.

### REFERENCES

1. Mirkin, C. A.; Letsinger, R. L.; Mucic, R. C.; Storhoff, J. J., A DNA-based method for rationally assembling nanoparticles into macroscopic materials. Nature 1996, 382, 607.
2. Cutler, J. I.; Auyeung, E.; Mirkin, C. A., Spherical nucleic acids. J. Am. Chem. Soc. 2012, 134, 1376.
3. Choi, C. H.; Hao, L.; Narayan, S. P.; Auyeung, E.; Mirkin, C. A., Mechanism for the endocytosis of spherical nucleic acid nanoparticle conjugates. Proc. Natl. Acad. Sci. U.S.A. 2013, 110, 7625.
4. Banga, R. J.; Chernyak, N.; Narayan, S. P.; Nguyen, S. T.; Mirkin, C. A., Liposomal spherical nucleic acids. J. Am. Chem. Soc. 2014, 136, 9866.
5. Radovic-Moreno, A. F.; Chernyak, N.; Mader, C. C.; Nallagatla, S.; Kang, R. S.; Hao, L.; Walker, D. A.; Halo, T. L.; Merkel, T. J.; Rische, C. H.; Anantatmula, S.; Burkhart, M.; Mirkin, C. A.; Gryaznov, S. M., Immunomodulatory spherical nucleic acids. Proc. Natl. Acad. Sci. U.S.A. 2015, 112, 3892.
6. Guan, C.; Chernyak, N.; Dominguez, D.; Cole, L.; Zhang, B.; Mirkin, C. A., RNA-Based Immunostimulatory Liposomal Spherical Nucleic Acids as Potent TLR7/8 Modulators. Small 2018, 14, e1803284.
7. Ferrer, J. R.; Wertheim, J. A.; Mirkin, C. A., Dual Toll-Like Receptor Targeting Liposomal Spherical Nucleic Acids. Bioconjug. Chem. 2019, 30, 944.
8. Wang, S.; Qin, L.; Yamankurt, G.; Skakuj, K.; Huang, Z.; Chen, P. C.; Dominguez, D.; Lee, A.; Zhang, B.; Mirkin, C. A., Rational vaccinology with spherical nucleic acids. Proc. Natl. Acad. Sci. U.S.A. 2019, 116, 10473.
9. Yamankurt, G.; Berns, E. J.; Xue, A.; Lee, A.; Bagheri, N.; Mrksich, M.; Mirkin, C. A., Exploration of the nanomedicine-design space with high-throughput screening and machine learning. Nat Biomed Eng 2019, 3, 318.
10. Jin, R.; Wu, G.; Li, Z.; Mirkin, C. A.; Schatz, G. C., What controls the melting properties of DNA-linked gold nanoparticle assemblies? J. Am. Chem. Soc. 2003, 125, 1643.
11. Xu, X.; Rosi, N. L.; Wang, Y.; Huo, F.; Mirkin, C. A., Asymmetric functionalization of gold nanoparticles with oligonucleotides. J. Am. Chem. Soc. 2006, 128, 9286.
12. Kim, Y.; Macfarlane, R. J.; Jones, M. R.; Mirkin, C. A., Transmutable nanoparticles with reconfigurable surface ligands. Science 2016, 351, 579.
13. Nam, J. M.; Stoeva, S. I.; Mirkin, C. A., Bio-bar-code-based DNA detection with PCR-like sensitivity. J. Am. Chem. Soc. 2004, 126, 5932.
14. Prigodich, A. E.; Lee, O. S.; Daniel, W. L.; Seferos, D. S.; Schatz, G. C.; Mirkin, C. A., Tailoring DNA structure to increase target hybridization kinetics on surfaces. J. Am. Chem. Soc. 2010, 132, 10638.
15. Prigodich, A. E.; Randeria, P. S.; Briley, W. E.; Kim, N. J.; Daniel, W. L.; Giljohann, D. A.; Mirkin, C. A., Multiplexed nanoflares: mRNA detection in live cells. Anal. Chem. 2012, 84, 2062.
16. Ming, X.; Laing, B., Bioconjugates for targeted delivery of therapeutic oligonucleotides. Adv Drug Deliv Rev 2015, 87, 81.
17. Krieg, A. M., Therapeutic potential of Toll-like receptor 9 activation. Nat. Rev. Drug Discov. 2006, 5, 471.
18. Klinman, D. M., Immunotherapeutic uses of CpG oligodeoxynucleotides. Nat. Rev. Immunol. 2004, 4, 249.
19. Vollmer, J.; Weeratna, R.; Payette, P.; Jurk, M.; Schetter, C.; Laucht, M.; Wader, T.; Tluk, S.; Liu, M.; Davis, H. L.; Krieg, A. M., Characterization of three CpG oligodeoxynucleotide classes with distinct immunostimulatory activities. Eur. J. Immunol. 2004, 34, 251.
20. Blazar, B. R.; Krieg, A. M.; Taylor, P. A., Synthetic unmethylated cytosine-phosphate-guanosine oligodeoxynucleotides are potent stimulators of antileukemia responses in naive and bone marrow transplant recipients. Blood 2001, 98, 1217.
21. Ashley, C. E.; Carnes, E. C.; Phillips, G. K.; Padilla, D.; Durfee, P. N.; Brown, P. A.; Hanna, T. N.; Liu, J.; Phillips, B.; Carter, M. B.; Carroll, N. J.; Jiang, X.; Dunphy, D. R.; Willman, C. L.; Petsev, D. N.; Evans, D. G.; Parikh, A. N.; Chackerian, B.; Wharton, W.; Peabody, D. S.; Brinker, C. J., The targeted delivery of multicomponent cargos to cancer cells by nanoporous particle-supported lipid bilayers. Nat. Mater. 2011, 10, 389.
22. Irvine, D. J., Drug delivery: One nanoparticle, one kill. Nat. Mater. 2011, 10, 342.
23. Kerkmann, M.; Costa, L. T.; Richter, C.; Rothenfusser, S.; Battiany, J.; Hornung, V.; Johnson, J.; Englert, S.; Ketterer, T.; Heckl, W.; Thalhammer, S.; Endres, S.; Hartmann, G., Spontaneous formation of nucleic acid-based nanoparticles is responsible for high interferon-alpha induction by CpG-A in plasmacytoid dendritic cells. J. Biol. Chem. 2005, 280, 8086.
24. de Titta, A.; Ballester, M.; Julier, Z.; Nembrini, C.; Jeanbart, L.; van der Vlies, A. J.; Swartz, M. A.; Hubbell, J. A., Nanoparticle conjugation of CpG enhances adjuvancy for cellular immunity and memory recall at low dose. Proc. Natl. Acad. Sci. U.S.A. 2013, 110, 19902.
25. Basu, S.; Wickstrom, E., Temperature and salt dependence of higher order structure formation by antisense c-myc and c-myb phosphorothioate oligodeoxyribonucleotides containing tetraguanylate tracts. Nucleic Acids Res. 1997, 25, 1327.
26. Förster, T., Zwischenmolekulare Energiewanderung und Fluoreszenz. Annalen der Physik 2006, 437, 55.
27. Meckes, B.; Banga, R. J.; Nguyen, S. T.; Mirkin, C. A., Enhancing the Stability and Immunomodulatory Activity of Liposomal Spherical Nucleic Acids through Lipid-Tail DNA Modifications. Small 2018, 14, 1702909.
28. Dearman, R. J.; Cumberbatch, M.; Maxwell, G.; Basketter, D. A.; Kimber, I., Toll-like receptor ligand activation of murine bone marrow-derived dendritic cells. Immunology 2009, 126, 475.
29. Lee, B. L.; Barton, G. M., Trafficking of endosomal Toll-like receptors. Trends Cell Biol. 2014, 24, 360.
30. Majer, O.; Liu, B.; Barton, G. M., Nucleic acid-sensing TLRs: trafficking and regulation. Curr. Opin. Immunol. 2017, 44, 26.
31. Schwartz, R. H., A cell culture model for T lymphocyte clonal anergy. Science 1990, 248, 1349.
32. Lancaster, G. I.; Khan, Q.; Drysdale, P.; Wallace, F.; Jeukendrup, A. E.; Drayson, M. T.; Gleeson, M., The physiological regulation of toll-like receptor expression and function in humans. J Physiol 2005, 563, 945.
33. June, C. H.; Bluestone, J. A.; Nadler, L. M.; Thompson, C. B., The B7 and CD28 receptor families. Immunol. Today 1994, 15, 321.
34. Madaan, A.; Verma, R.; Singh, A. T.; Jain, S. K.; Jaggi, M., A stepwise procedure for isolation of murine bone marrow and generation of dendritic cells. Journal of Biological Methods 2014, 1, e1.
35. Borlinghaus, R. T.; Kappel, C., HyVolution-the smart path to confocal super-resolution. Nat. Methods 2016, 13, I.
36. Manders, E. M.; Stap, J.; Brakenhoff, G. J.; van Driel, R.; Aten, J. A., Dynamics of three-dimensional replication patterns during the S-phase, analysed by double labelling of DNA and confocal microscopy. J. Cell Sci. 1992, 103, 857.
37. Manders, E. M. M.; Verbeek, F. J.; Aten, J. A., Measurement of co-localization of objects in dual-colour confocal images. J. Microsc. 1993, 169, 375.

The following examples are given merely to illustrate the present disclosure and not in any way to limit its scope.

### EXAMPLES

The following examples describe the synthesis and evaluation of spherical nucleic acids (SNAs) incorporating two physically and chemically distinct classes of oligonucleotides (ODNs) at programmed ratios. These SNAs are single entity agents that entered the same target cell at defined stoichiometries, and as such allow one to control important cell signaling and regulatory processes. To study the effect of sequence multiplicity within such structures, SNAs were synthesized consisting of a mixture of class A CpG and class B CpG, immunostimulatory ODNs that activate two different toll-like receptor 9 (TLR9) signaling pathways, each in a sequence-specific fashion. These dual-CpG SNAs exhibited high cellular uptake and co-delivery of the two ODNs, relative to mixtures of the linear ODN counterparts, and remained highly associated inside the cell over time. Furthermore, the dual-CpG SNAs augmented dendritic cell (DC) maturation, compared to the same amounts of oligonucleotides delivered in linear or SNA form but not conjugated to one another. Consequently, these structures constitute a platform for designing oligonucleotide-based combination therapeutics with highly tailorable activities.

The studies presented herein incorporate two physically-and chemically-distinct oligonucleotide sequences with onto the same SNA construct in controlled ratios. The resulting SNAs showed strong intracellular association of the two oligonucleotide sequences over extended periods of time, sequence-independent intracellular trafficking, and sequence- and ratio-dependent downstream activations, in addition to the known advantageous SNA properties of superior cellular uptake and enhanced activation as compared to their linear counterparts. The findings described herein provide vital insights about the design of SNAs as a platform for the delivery of multifunctional therapeutics.

To study the effect of sequence multiplicity within such structures, SNAs consisting of two physically (e.g., structural features) and chemically (e.g., backbone chemistry) distinct oligonucleotide sequences were synthesized with class A CpGs and class B CpGs, which are immunostimulatory ODNs that activate two different toll-like receptor 9 (TLR9) signaling pathways, each in a sequence-specific fashion. These dual-CpG SNAs exhibited high cellular uptake and co-delivery of the two ODNs, relative to mixtures of the linear ODN counterparts, and remained highly associated inside the cell over time. Furthermore, the dual-CpG SNAs augmented dendritic cell maturation, compared to the same amounts of oligonucleotides delivered in linear or SNA form but not conjugated to one another. Consequently, these structures constitute a platform for designing oligonucleotide-based combination therapeutics with highly tailorable activities.

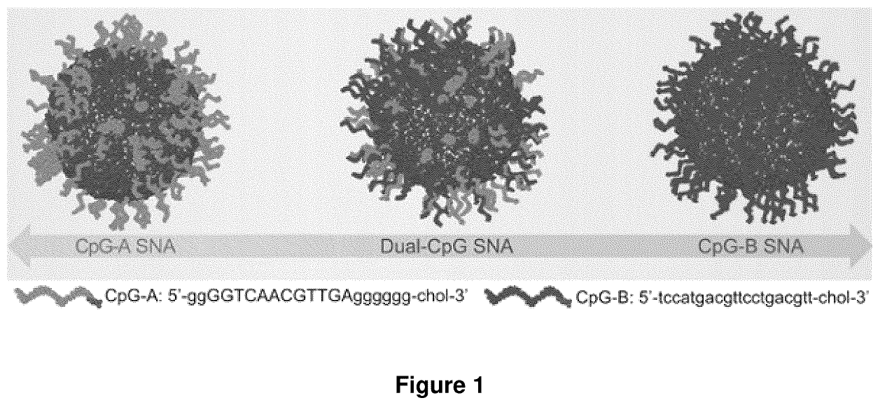

Delivering multiple entities on the same particle to immune cells is beneficial over administration of simple mixtures of the individual components, because the nanoparticle scaffold allows for delivery of programmed ratios of each component to the same target cell.²¹⁻²² In this study, two physically (e.g., structural features) and chemically (e.g., backbone chemistry) distinct oligonucleotide sequences, CpG-A (ODN 1585) and CpG-B (ODN 1826), were incorporated into LSNAs (FIG. 1) to study the intracellular trafficking of the SNAs and their downstream activation of DCs.

### Example 1

### Materials and Methods

1,2-dioleoyl-sn-glycero-3-phosphocholine (DOPC) was purchased from Avanti Polar Lipids as a solution in chloroform. TLR9 ligands CpG ODN 1585 (Class A), 1826 (Class B), and their corresponding GpC control sequences were synthesized on an ABI 3900 DNA oligo synthesizer with all reagents purchased from Glen Research (See below).

Synthesis of oligonucleotides. Oligonucleotides were synthesized using an ABI 3900 DNA oligo synthesizer on a standard controlled pore glass (GPG) solid phase support following the manufacturer's protocol. All modified phosphoramidites were purchased from Glen Research. The products were purified by reverse phase high-performance liquid chromatography (HPLC). Syntheses were verified by matrix-assisted laser desorption/ionization-time-of-flight mass spectrometry (MALDI-TOF), and the concentrations were determined by UV-visible spectroscopy. A complete list of oligonucleotides synthesized can be found in Table 1.

TABLE 1
List of oligonucleotide sequences used herein.

| Strand | DNA sequence from 5′ to 3′ | SEQ ID NO: |
|---|---|---|
| ODN 1585 (CpG-A)-chol | ggGGTCAACGTTGAgggggg-(Sp18)₂-chol | 2 |
| ODN 1826 (CpG-B)-chol | tccatgacgttcctgacgtt-(Sp18)₂-chol | 3 |
| Linear CpG-A | ggGGTCAACGTTGAgggggg | 4 |
| Linear CpG-B | tccatgacgttcctgacgtt | 5 |
| Fluorescein-CpG-A-chol | ggGGTCAACGTTGAgggggg-(Sp18)-FAM-(Sp18)-chol | 6 |
| Cy3-CpG-B-chol | tccatgacgttcctgacgtt-(Sp18)-Cy3-(Sp18)-chol | 7 |
| Cy5-CpG-B-chol | tccatgacgttcctgacgtt-(Sp18)-Cy5-(Sp18)-chol | 8 |
| Linear fluorescein-CpG-A | ggGGTCAACGTTGAgggggg-FAM | 9 |
| Linear Cy5-CpG-B | tccatgacgttcctgacgtt-Cy5 | 10 |

Notations and abbreviations:
1. Upper-case letter bases denote phosphodiester backbone, and lower-case letter bases denote phosphorothioate backbone. Underlined portion indicates palindrome.
2. “chol” refers to 1-dimethoxytrityloxy-3-O-(N-cholesteryl-3-aminopropyl)-triethyleneglycol-glyceryl-2-O-succinoyl-long chain alkylamino-CPG (3′-Cholesteryl-TEG CPG).
3. “Sp18” refers to 18-O-dimethoxytritylhexaethyleneglyco1,1-[(2-cyanoethyl)-(N,N-diisopropyl)]-phosphoramidite (Spacer phosphoramidite 18).
4. “FAM” refers to 1-dimethoxytrityloxy-2-(N-thiourea-(di-O-pivaloyl-fluorescein)-4-aminobutyl)-propyl-3-O-(2-cyanoethyl)-(N,N-diisopropyl)-phosphoramidite (Fluorescein phosphoramidite).
5. “Cy3” refers to 1-[3-(4-monomethoxytrityloxy)propyl]-1′-[3-[(2-cyanoethyl)-(N,N-diisopropyl) phosphoramidityl]propyl]-3,3,3′,3′-tetramethylindocarbocyanine chloride (Cyanine 3 phosphoramidite).
6. “Cy5” refers to 1-[3-(4-monomethoxytrityloxy)propyl]-1′-[3-[(2-cyanoethyl)-(N,N-diisopropyl) phosphoramidityl]propyl]-3,3,3′,3′-tetramethylindodicarbocyanine chloride (Cyanine 5 phosphoramidite).

Cell lines. Bone marrow-derived dendritic cells (BMDCs) were collected from C57BL/6 wildtype mice and processed following the literature protocol³⁴, and the cells were cultured in RPMI 1640 medium (Gibco) supplemented with 10% fetal bovine serum (FBS, Atlanta Biologicals), 100 U/mL penicillin-streptomycin (pen-strep, Gibco), and 20 ng/ml recombinant mouse granulocyte-macrophage colony stimulating factor (GM—CSF, eBioScience). JAWSII, an immortal murine BMDC cell line, was purchased from ATCC (CRL-11904) and cultured in alpha minimum essential medium (Gibco) supplemented with 1 mM sodium pyruvate (Gibco), 5 ng/ml GM—CSF, 20% FBS, and 100 U/mL pen-strep. RAW Dual cells, a RAW 264.7 murine macrophage reporter cell line for IRF and nuclear factor κB (NF-κB), was purchased from InvivoGen and cultured in Dulbecco's Modified Eagle Medium, 10% FBS, 100 U/mL pen-strep, 100 μg/mL Normocin (InvivoGen), and 200 μg/mL Zeocin (InvivoGen). Reagents for reporter cell line assays (Quanti-Blue and Quanti-Luc) were also purchased from InvivoGen.

Preparation of liposomal SNAs (LSNAs). In a typical synthesis, 4 mL of a chloroform solution of DOPC lipid (25 mg/mL) was transferred into a glass vial, and the lipid dried under nitrogen gas for 30 minutes and stored in vacuo overnight to completely remove organic solvent and generate a thin lipid film. The film was then rehydrated in 10 mL of PBS. The resulting suspension was vortexed for 30 seconds and sonicated for 30 minutes. Following five freeze-thaw cycles, the suspension underwent serial extrusions in 200-nm, 100-nm, 80-nm, and 50-nm track-etch membranes to obtain uniform-size liposomes. Liposomes were concentrated using 50K MWCO MicroKros tangential flow filtration (Spectrum Labs), and the final concentration was measured using the phosphatidylcholine assay kit (Sigma). To functionalize oligonucleotides onto the surface of liposomes, 100 μM 3′-cholesterol-functionalized oligonucleotides were mixed with a predetermined amount of liposomes, calculated following Table 2. Samples were held overnight at 37° C. under shaking at 500 rpm. Formulation of CpG and dual-CpG SNAs is shown in Table 2.

TABLE 2
Dual-CpG SNA composition.

| | | CpG-A: | CpG-A | | CpG-B: | CpG-B | liposome |
|---|---|---|---|---|---|---|---|
| | CpG-A | liposome | conc. | CpG-B | liposome | conc. | conc. |
| Sample # | mol % | (μM/μM) | (μM) | mol % | (μM/μM) | (μM) | (μM) |
| 1 | 100% | 150 | 100 | 0% | 0 | 0 | 0.667 |
| 2 | 90% | 135 | 90 | 10% | 7.5 | 10 | 0.733 |
| 3 | 70% | 105 | 70 | 30% | 22.5 | 30 | 0.867 |
| 4 | 50% | 75 | 50 | 50% | 37.5 | 50 | 1.000 |
| 5 | 30% | 45 | 30 | 70% | 52.5 | 70 | 1.133 |
| 6 | 10% | 15 | 10 | 90% | 67.5 | 90 | 1.267 |
| 7 | 0% | 0 | 0 | 100% | 75 | 100 | 1.333 |

Materials characterization. To assess CpG-A incorporation into SNAs, agarose gel electrophoresis was performed using 1% agarose (Sigma) with SYBR Safe DNA gel stain in 0.5× Tris/Borate/EDTA (TBE) (ThermoFisher Scientific) under 120 V for 1 hour. To assess dual-CpG loading, fluorophore-labeled oligonucleotides (FAM-CpG-A and Cy5-CpG-B) were used, and agarose gel electrophoresis was performed using 0.5% agarose in 1× TBE without further staining. Dynamic light scattering (DLS) and zeta potential measurements were performed using a Malvern Zetasizer Nano with approximately 10 nM samples by particle. Cryogenic transmission electron microscopy (CryoTEM) was performed with a Hitachi HT7700 TEM with a

Gatan cryo-transfer holder, and imaging was performed under a 120-kV accelerating voltage. CryoTEM samples were prepared by loading 4 μL of sample (100 μM CpG SNA stock solution or 1.33 μM liposome solution) onto 300-mesh copper TEM grids with lacey carbon films (Electron Microscopy Sciences) using a FEI Vitrobot Mark IV with its chamber equilibrated at 4° C. and 100% humidity. The samples were blotted for 5 s and plunged into liquid ethane before they were transferred and stored in liquid nitrogen. Förster resonance energy transfer (FRET) measurements were performed using a Synergy plate reader (Biotek) with 10 nM samples by oligonucleotides. FRET efficiency was calculated as:

$$E = 1 - \frac{{F}_{DA}}{{F}_{D}}$$

where F_{DA} and F_{D} are the donor fluorescence intensities with and without an acceptor, respectively.

Confocal microscopy and image analysis. 2×10⁴ JAWSII cells in 0.5 mL cell phenol red-free culture medium were plated on gelatin-coated 15-mm coverslips in 24-well plates overnight and incubated with 5 μM SNA by CpG oligonucleotide concentration. Fluorescein-labeled CpG-A ODN and/or Cy5-labeled CpG-B ODN were used, and the high concentration was used to ensure detectable signal after a prolonged time. To track the same amount of SNAs trafficking over time, all cells were treated for 30 minutes. For the 30 minute time points in the intracellular trafficking studies, cells were incubated with media containing either SNAs or linear CpG ODNs for 30 minutes, then washed, removed from media, fixed, and stained. For all other time points, cells were incubated in SNA or CpG-containing media for 30 minutes, washed twice, and incubated with fresh media for the additional amount of time prior to fixation and staining. At the end of the treatment, coverslips were incubated in 4% paraformaldehyde in PBS (diluted from a 32% aqueous solution from Electron Microscopy Sciences) for 15 minutes for fixation and 0.2% Triton-X in PBS (diluted from Triton-X from Sigma) for 10 minutes for permeation.

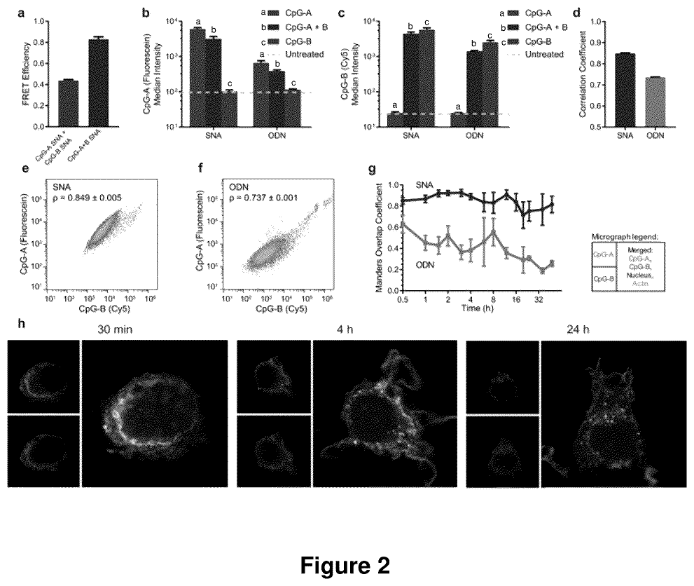

For the CpG association study (FIG. 2), the cells were incubated in Image-iT™ FX Signal Enhancer (ThermoFisher Scientific) for 30 minutes and then in Alexa Fluor 568 Phalloidin for 30 minutes. Phalloidin labels the actin of the cytoskeleton, which here serves as the confirmation of DC structure (the dendrite growth over time may also serve as confirmation of dendritic cell maturation, though it is difficult to quantify). For the intracellular trafficking study, coverslips were incubated in 2% bovine serum albumin (BSA) and 0.2% Triton-X in PBS for 1 hour for blocking. Rabbit anti-EEA1 (ABclonal), rabbit anti-Rab9 (Abclonal), rabbit anti-LAMP1 (Abcam), and rabbit anti-LC3 (Sigma-Aldrich) primary antibodies diluted 1:1000 in 1% BSA, 0.1% Triton-X in PBS were used for early endosome, late endosome, lysosome, and autophagosome labeling, respectively. Alexa Fluor 568-labeled goat anti-rabbit IgG H&L secondary antibody (Abcam) diluted 1:1000 in 1% BSA, 0.1% Triton-X in PBS was used to fluorescently tag the intracellular organelle markers. All treatment and staining steps used 0.4 mL volumes, and coverslips were washed twice using 1 mL PBS or thrice using PBS-Triton-X (for antibody washes) after each step. After staining, coverslips were mounted onto glass slides using ProLong Glass with NucBlue nuclear stain (ThermoFisher Scientific) and cured overnight. Confocal microscopy was performed using Leica TCS SP8 LSCM with hybrid detectors (HyD) and HyVolution.³⁵

Data processing and colocalization calculations were performed using the Leica Application Suite X (for HyVolution) and an in-house Matlab script. A total of 25 cells were included for the analysis for each sample. Images of individual detection channels were exported as grayscale maps and denoised using deep neural network. Z-stack layers of whole cells were arranged into a three-dimensional array as pseudo-3D reconstruction of the cell image for each channel. Colocalization analysis provides a quantitative means to evaluate the qualitative confocal image data. Manders overlap and colocalization coefficients³⁶⁻³⁷ were used in this study to analyze the colocalization of color pairs from the confocal images. For analyzing the association of the two CpG ODNs, the Manders overlap coefficient, a parameter describing the degree of overlap between two components in an image, was calculated. The Manders overlap coefficient, a derivative of the Pearson's correlation coefficient, is defined as:

$$r = \frac{{\sum }_{i} {a}_{i} \cdot {b}_{i}}{\sqrt{{\sum }_{i} {( {a}_{i} )}^{2} \cdot {\sum }_{i} {( {b}_{i} )}^{2}}}$$

where aᵢ and bᵢ are the grey-scale values of the individual voxels i of the color components a and b, which in this case, were the fluorescein and Cy5 signals. For analysis of colocalization of CpG ODN in the organelles, the Manders colocalization coefficient was used which is defined by:

$${M}_{ab} = \frac{{\sum }_{i} {a}_{i , {b}_{i} > 0}}{{\sum }_{i} {a}_{i}}$$

so that Mab measures the fluorescence fraction of a colocalized with b, which in this case was the ODN signal (fluorescein and/or Cy5) colocalized in the organelle (Alexa Fluor 568). This coefficient was useful since it does not consider the pixels of the second signal (organelles) that do not overlap with the first signal (ODN). Both Manders overlap and colocalization coefficients are signal intensity independent, which made them advantageous in this study to compare markers that may have different signal intensities caused by different uptake amount (SNA vs. linear oligonucleotides), CpG signal decrease over time, and difference in organelle antibody labeling.

Flow Cytometry. All antibodies and staining buffers were purchased from BD Biosciences, and all antibodies were mixed and diluted to 1:200 in staining buffer. For the uptake study, 3×10⁵ cells were treated with fluorescein-labeled CpG-A and/or Cy5-labeled CpG-B oligonucleotides with a total oligonucleotide concentration of 1 μM in 1.2 mL microtubes. After a 30-minute incubation at 37° C., cells were washed three times with 1 mL PBS and stained with 100 μL of antibody solution containing rat anti-mouse CD11b and hamster anti-mouse CD11c. Pearson correlation calculations were performed using fluorescein (G) and Cy5 (R) signals of each cell into the formula:

$$ρ ( G , R ) = \frac{1}{N - 1} {\sum }_{i = 1}^{N} ( \frac{{G}_{i} - {μ}_{G}}{{σ}_{G}} ) ( \frac{{R}_{i} - {μ}_{R}}{{σ}_{R}} )$$

where μ_{G} and σ_{G} are the mean and standard deviation of G, respectively, and μ_{R} and σ_{R} are the mean and standard deviation of R, respectively. For the dendritic cell activation study, 3×10⁵ cells were incubated with 200 μL of 1 μM CpG (linear or SNA) containing media for 24 hours in 1.2 mL microtubes. The cells were then washed three times with 1 mL PBS and stained with 100 μL of antibody solution containing rat anti-mouse CD11b, hamster anti-mouse CD11c, hamster anti-mouse CD80, and rat anti-mouse CD86 for 15 minutes at 4° C. The cells were then fixed with 3.7% paraformaldehyde in PBS for 20 minutes at room temperature. Flow cytometry was completed with a BD LSRFortessa flow cytometer system. 30,000 events were collected for each sample, and DCs gated with CD11c+ population. Color compensation was performed using the AbC total antibody compensation bead kit (ThermoFisher Scientific). Data analysis was performed using FlowJo and Matlab.

Cytokine and IFN measurements. Cytokine and IFN release were measured using Luminex 200. Assays were performed using mouse magnetic Luminex assay kits purchased from R&D Systems for cytokines and ThermoFisher Scientific for IFN-α. 2×10⁵ cells were treated with 200 μL media containing 100 nM CpG (linear or SNA) in a round-bottom 96-well plate for 24 hours, and after centrifugation for 5 minutes at 1,000 rpm, the cell supernatants were collected and transferred to the Luminex assay plate without further dilution. Sample processing and measurements were performed following the manufacturer's specific protocols. Data analysis was performed using Milliplex Analyst software.

### Results and Discussion

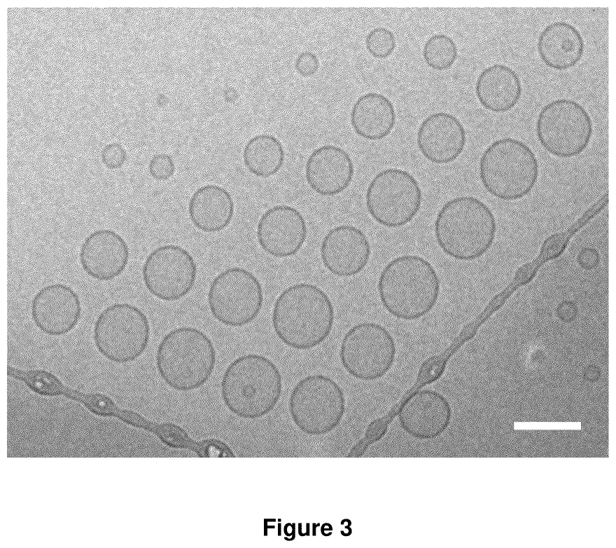

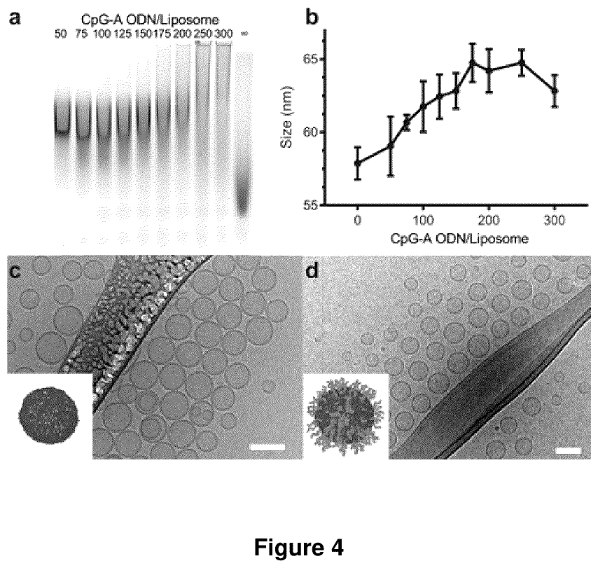

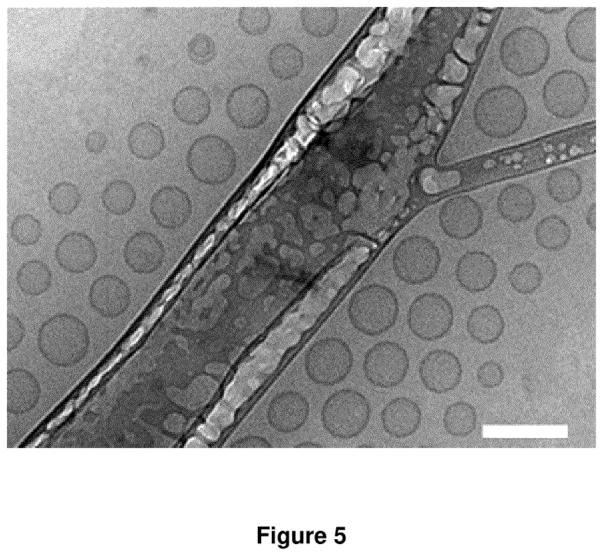

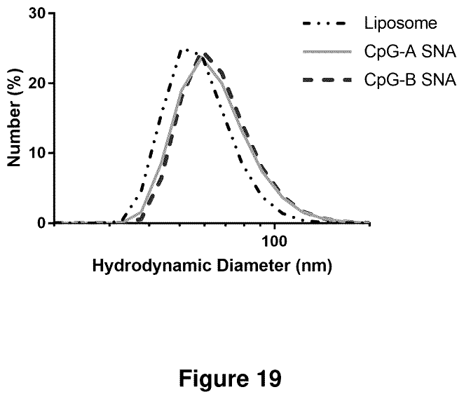

To generate SNAs, 3′-cholesterol-terminated oligonucleotides and 50-nm 1,2-dioleoyl-sn-glycero-3-phosphocholine (DOPC) liposomes were mixed in a predetermined ratio to achieve the desired CpG ODN loading on the SNA scaffold.^{4, 8} First, single-component SNAs, comprised of either all CpG-A or all CpG-B DNA, were synthesized and characterized to understand the impact of sequence and secondary DNA structure on the DNA loading of SNAs. In particular, CpG-B SNAs (FIG. 3) have been well-characterized and studied as cancer immunotherapeutics with a maximal loading of 75 CpG-B ODN per 50 nm liposome.^{5, 8-9} Conversely, CpG-A ODN has been proposed to have nanoparticle-like properties because it contains a poly(G) sequence that forms G-quadruplexes that can potentially stabilize the palindrome sequence.²³⁻²⁴ Thus, CpG-A is rarely incorporated into nanomaterials and has never been used as a component of SNAs. However, these higher-order structures of CpG-A are salt- and temperature-dependent;^{23, 25} thus, it was hypothesized that CpG-A aggregation could be circumvented by incorporating CpG-A into SNAs during the SNA assembly process (as is shown herein). Therefore, the oligonucleotide content of CpG-A in SNAs was first studied and it was determined through agarose gel electrophoresis that up to 150 cholesterol-terminated CpG-As could be inserted into 50-nm DOPC liposomes (FIG. 4a). This observation was confirmed by the size increase trend, as function of oligonucleotide equivalents, determined by dynamic light scattering (DLS, FIGS. 4b and 19). Cryogenic transmission electron microscopy (CryoTEM) analysis revealed that unfunctionalized liposomes pack tightly together, presumably due to the absence of negatively charged surface ligands and therefore minimized electrostatic repulsion between particles (FIG. 4c). However, when the liposomes were transformed into SNAs through their external modification with CpG-A, there was a marked increase in interparticle spacing (FIG. 4d), a trend that was also observed with CpG-B liposomal SNAs (FIG. 3). For example, CpG-A SNA interparticle spacing increased with increased oligonucleotide loading from 21.0±3.5 nm (50 strands/SNA, FIG. 5) to 27.7±3.7 nm (150 strands/SNA, FIG. 4d). These results not only showed that higher oligonucleotide loading density lead to higher particle surface coverage, but also created more radially oriented ODNs within the SNA structure. When the DNA: liposome ratio exceeded 150:1, an upward shift in mobility by gel electrophoresis was observed (FIG. 4a), indicating a significant size increase in the population, presumably due to aggregation of the SNAs caused by sequence self-complementarity (as discussed herein below). To avoid aggregation-specific effects on cellular uptake and downstream immune activation, SNAs incorporating 150 strands of CpG-A ODN per particle were used in further studies.

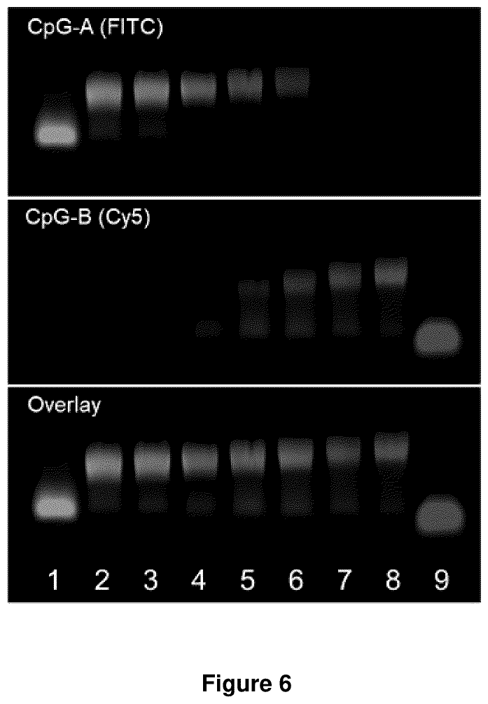

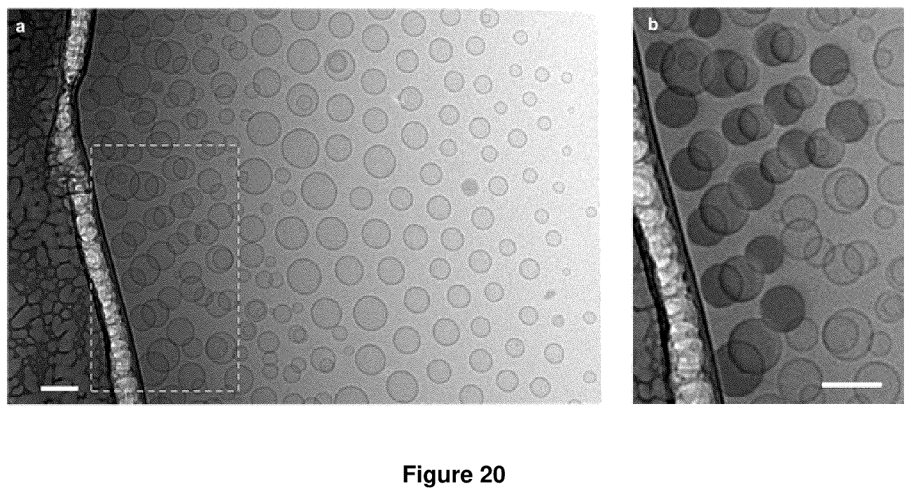

SNAs incorporating both classes of CpG ODN on the same nanoparticle scaffold were next synthesized by incorporating CpG-A and CpG-B into unfunctionalized DOPC liposomes at defined ratios (Table 2), based on the determined saturation limits of each single-component SNA. Agarose gel electrophoresis revealed that both CpG classes were incorporated into SNAs (FIG. 6). To confirm the successful dual functionalization of SNAs with both CpG ODN, Förster resonance energy transfer (FRET), the energy transfer process from one excited fluorophore to an acceptor fluorophore (such as from fluorescein to Cy3) when donor and acceptor are within the Forster radius, was employed.²⁶⁻²⁷ Dual-CpG SNAs functionalized with 50% fluorescein-labeled CpG-A and 50% Cy3-labeled CpG-B showed a significantly higher FRET efficiency as compared to a 50:50 mixture of two distinct sets of SNAs, each incorporating one fluorophore-labeled class of CpG ODN (FIG. 2a). Such significantly higher FRET efficiency indicated that the two CpG ODN classes were in closer proximity when functionalized on the same SNA compared to when they were functionalized on separate SNAs, reflecting successful dual sequence functionalization on the same SNA. The dual-CpG SNAs were also evaluated by CryoTEM analysis (FIG. 20).

To evaluate the uptake and immunogenicity of dual-CpG SNAs, mouse bone marrow-derived dendritic cells (DCs), a TLR9-presenting cell line widely utilized for in vitro immune stimulation studies using CpG ODNs, were used as a model system.²⁸ DCs incubated with SNAs showed significantly higher uptake as compared to cells treated with linear ODNs (FIG. 2b-c) after 30 minutes. Dual-CpG SNAs showed similar uptake to SNAs functionalized with a single class of CpG ODN (FIGS. 2b-c and 7) on a per particle basis. As expected, the fluorescence of cells treated with dual-CpG SNAs incorporating fluorescein-labeled CpG-A and Cy5-labeled CpG-B at a 50:50 mol ratio was roughly half the intensity of cells treated with single-component SNAs, as each CpG component encompassed half of the total CpG ODN incorporated into the dual-CpG SNA scaffold. Furthermore, cells treated with 50:50 dual-CpG SNAs showed a Pearson correlation coefficient (ρ), a measure of the linear correlation between CpG-A uptake and CpG-B uptake, greater than 0.8 (ρ=0.849±0.005), indicating that dual-CpG SNAs provided better co-delivery of the two components as compared to a 50:50 mixture of the two linear CpG ODNs (not on SNAs) which did not show a strong correlation of co-delivery (FIGS. 2d-f and 7). Confocal microscopy revealed that even after extended periods (e.g., 48 hours), the fluorescence from the two labeled CpG components of 50:50 dual-CpG SNAs still strongly overlapped, quantified by high Manders overlap coefficients, while fluorescence from cells treated with a 50:50 mixture of both linear labeled CpG ODNs showed little overlap (FIGS. 2g-h and 8).

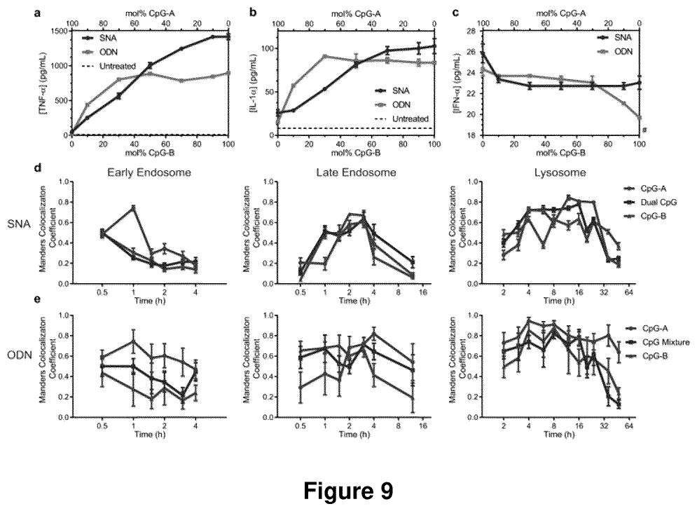

Although CpG ODNs activate TLR9 and lead to the secretion of cytokines and interferons, different classes of CpG ODN induce different immune responses, due in part to different intracellular trafficking of the oligonucleotides.²⁹⁻³⁰ After entering cells, CpG-A binds to TLR9 preferentially in the early endosome, which activates the interferon regulatory factor 7 (IRF7) pathway and leads to the secretion of type I IFN (e.g., IFN-α). In contrast, CpG-B activates TLR9 primarily in the late endosome, where the nuclear factor κB (NF-κB) pathway is predominately activated and secretes proinflammatory cytokines. The detailed mechanisms of different activation pathways are still not completely understood, but it is proposed that both the sequence of the oligonucleotides and the pH of the intracellular organelles may contribute. Both factors likely impact the morphology of the CpG-TLR9 complex and consequently the extra-organelle portion of TLR9 responsible for downstream activation. Thus, a series of dual-CpG SNAs with different ratios of CpG-A to CpG-B loading were generated, ranging from 0% CpG-B (100% CpG-A) to 100% CpG-B (0% CpG-A) to study the effects of codelivery on downstream activation, with oligonucleotide loading confirmed by agarose gel electrophoresis (FIG. 6). After incubating DCs with 1 μM SNAs (by total CpG concentration) for 24 hours, it was found that high CpG-B loading promoted cytokine secretion (FIGS. 9a-b and 10), indicative of activation of the NF-κB pathway, while high CpG-A loading led to high IFN-α production, indicative of activation of the IRF7 pathway (FIG. 9c). Further, cells treated with single-component CpG-A SNAs or CpG-B SNAs stimulated higher cytokine and interferon production as compared to their linear counterparts. Interestingly, although cytokine secretion increased steadily with increasing CpG-B loading in SNAs, the linear ODN mixture reached a maximum level of cytokine secretion at 30% CpG-B, with no enhanced cytokine production observed at higher ratios of CpG-B to CpG-A (FIG. 9a-b). Similar behavior was observed for IFN-α production by linear ODN mixtures as a function of CpG-A ratios (FIG. 9c).

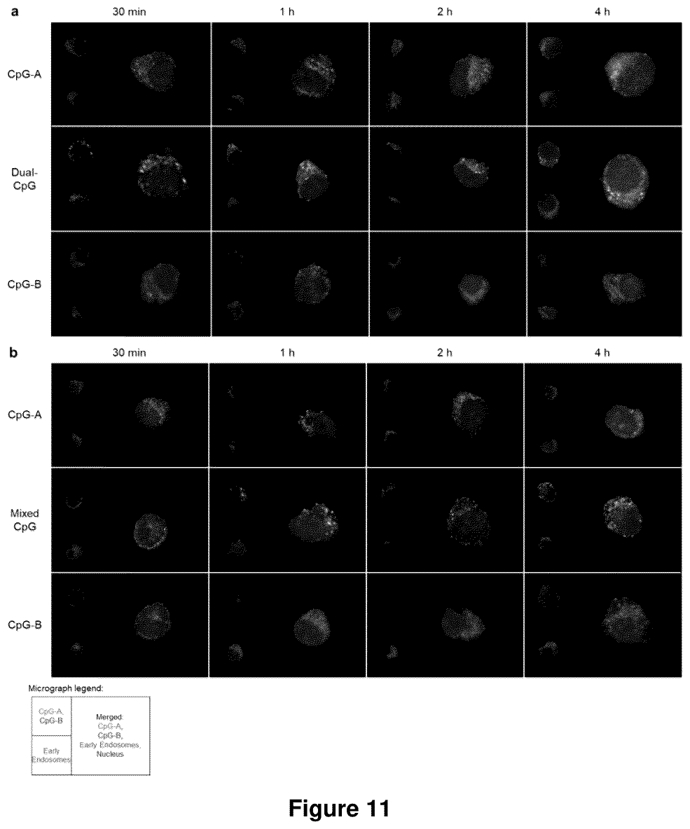

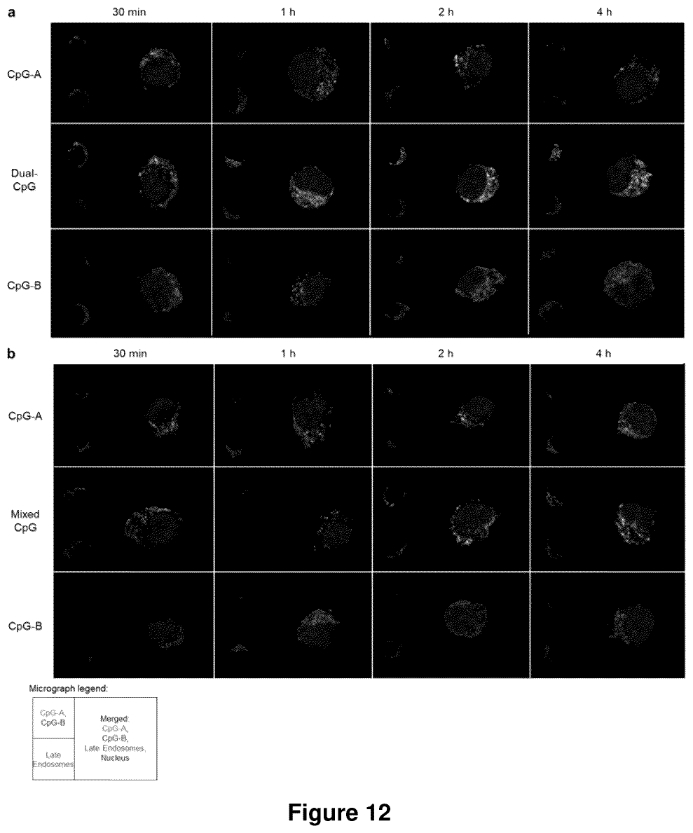

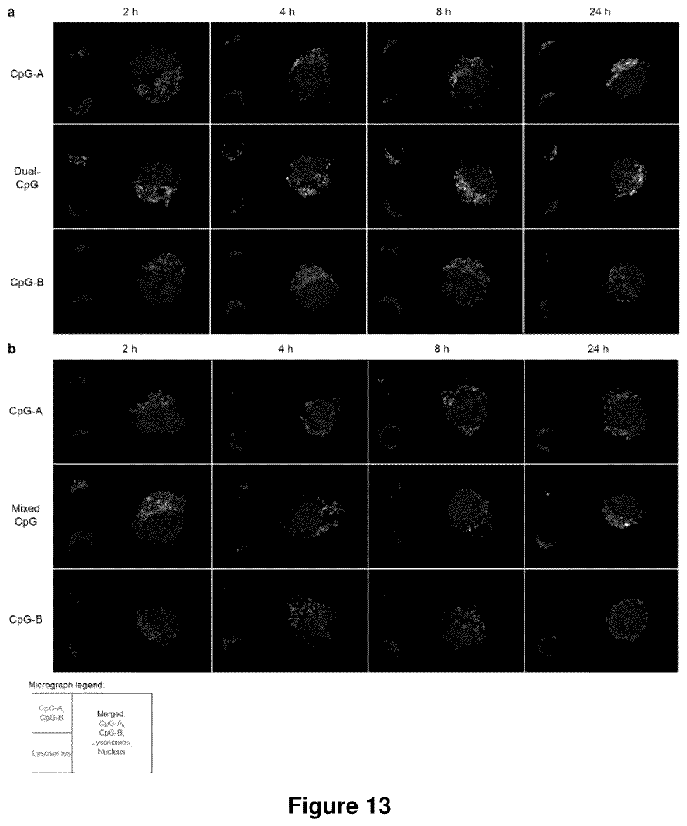

It was hypothesized that the differences in immune activation by SNAs as compared to linear ODNs was a result of the difference in intracellular trafficking of the two constructs. In the case of linear CpG ODNs, each component traveled independently to its preferred intracellular pathway for downstream activation, since the two components were not structurally associated. However, CpG-A and CpG-B were coupled onto the same entity in dual-CpG SNAs and remained highly associated over time (FIG. 2g). Thus, CpG-A and CpG-B were trafficked to the same location, leading to only a portion of each CpG type delivered to its preferred intracellular organelle for TLR9 activation. To confirm this hypothesis, the intracellular trafficking of different SNAs was studied using confocal microscopy (FIGS. 11-13), as well as the localization of CpG in intracellular organelles (FIGS. 9d-e, and 14). It was found that the trafficking of CpG-A and CpG-B ODNs was primarily dictated by the SNA scaffold, with sequence identity having minimal effect on intracellular trafficking when the ODNs were on an SNA. Conversely, the trafficking of mixtures of linear ODNs was dictated by each ODN sequence identity. CpG SNAs, regardless of sequence, were trafficked to early endosomes, late endosomes, and lysosomes at approximately the same rates and on the same timescales (FIG. 9d). In general, SNAs colocalized with the early endosome after uptake, followed by a rapid decrease with a concomitant increase of colocalization with the late endosome. There was only 0.5 hour difference between CpG-A and CpG-B SNAs that colocalized with the early endosome, and all SNAs showed colocalization with the late endosome with a peak at approximately 2-3 hours. Colocalization of SNAs in lysosomes steadily increased, reaching a plateau at 4 hours which was maintained up to 24 hours. On the contrary, mixtures of linear CpG-A and CpG-B ODNs were trafficked very differently (FIG. 9e). After uptake, linear CpG-A ODN maintained high colocalization with the early endosome, where CpG-A/TLR9 binding is preferred, with a slight decrease in colocalization over time. In contrast, linear CpG-B ODN showed high colocalization with the late endosome at early time points, indicating very rapid traveling to late endosomes, where CpG-B/TLR9 binding is favored. These results will guide the design of SNAs for preferentially delivering various oligonucleotide sequences to specific intracellular organelles following this particular spatiotemporal path, which is otherwise impossible to control for the corresponding linear ODN counterparts.

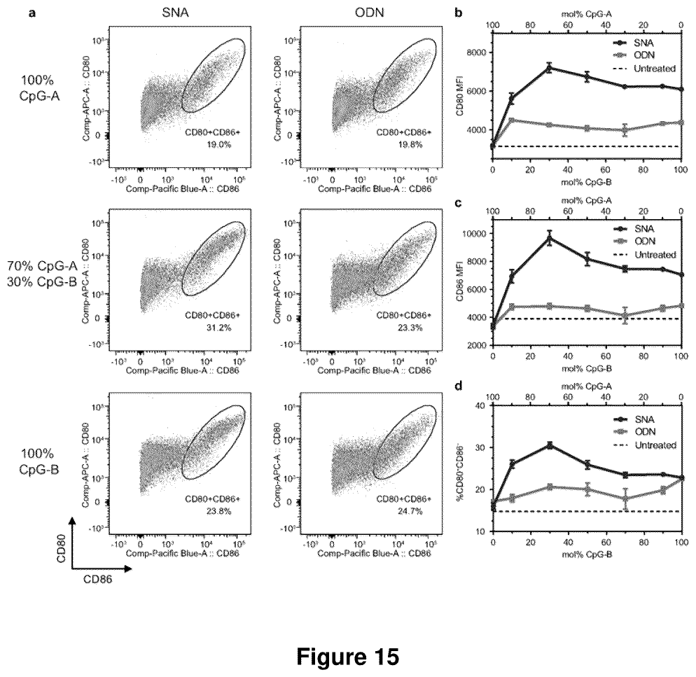

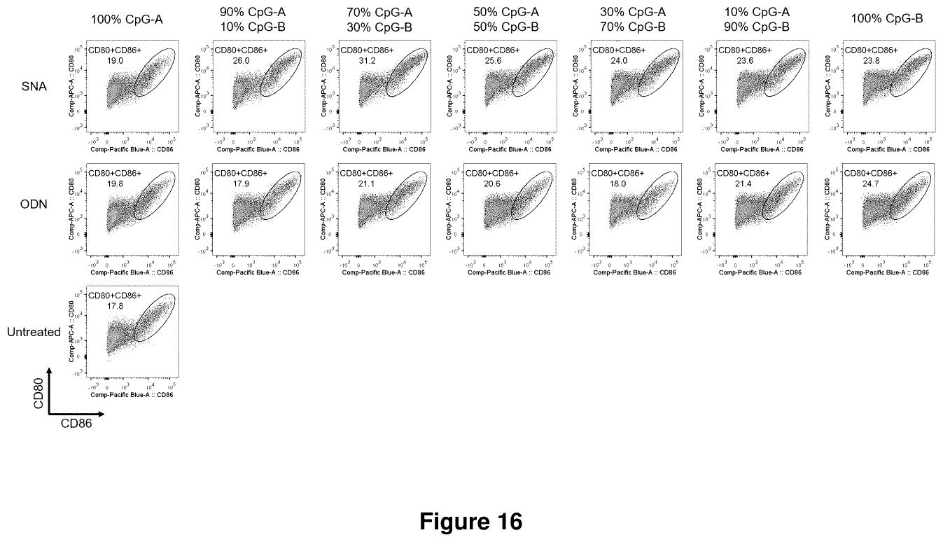

It was further hypothesized that maximum DC activation could be achieved by codelivering an intermediate ratio of CpG-A and CpG-B on the same SNA. Costimulation markers are antigen non-specific signaling proteins that are necessary for antigen-presenting cells (e.g., DCs) to activate T cells.³¹ In addition to cytokine, chemokine, and interferon productions, TLR9 activation also leads to the upregulation of CD80 and CD86 (also known as B7-1 and B7-2, respectively), two proteins that can bind to CD28 on T cells for critical costimulation and T cell activation.³²⁻³³ After incubating DCs with dual-CpG SNAs for 24 hours, SNAs at different CpG-A/B ratios demonstrated almost consistently superior performances to incubation with linear CpG ODNs at the same ratios (FIGS. 15 and 16). Expression of CD80 and CD86 peaked at 70 mol % CpG-A and 30 mol % CpG-B ratio on SNAs, and the percentage of the CD80⁺/CD86+ double-positive population was also the highest at this CpG ratio. These results showed that dual-CpG SNAs with controlled oligonucleotide ratios can achieve maximal DC maturation, potentially because of the unique intracellular trafficking pathway taken by SNAs, allowing cells to take advantage of activation by both CpG classes. After SNAs enter cells, a relatively higher amount of CpG-A than CpG-B was needed to effectively bind TLR9 in the early endosome and induce significant downstream activation, because SNAs only remain in the early endosome for a brief time (FIG. 9d). Conversely, a relatively lower amount of CpG-B was needed because the SNAs remain colocalized with the late endosome for extended periods, where CpG-B is processed. It was found that 70 mol % CpG-A and 30 mol % CpG-B was the optimal ODN ratio on dual-CpG SNAs to achieve maximal activation (FIG. 15).

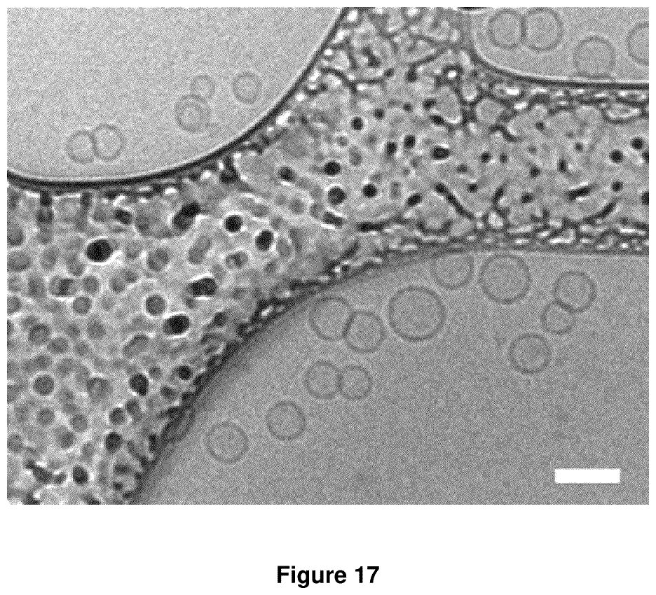

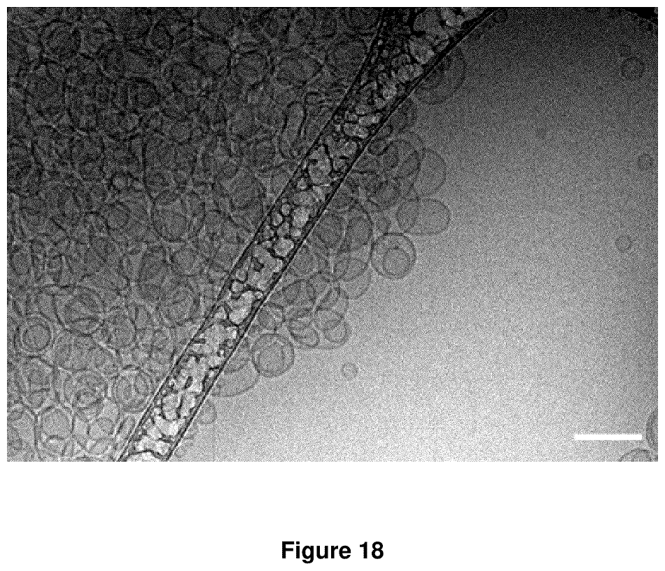

CpG-A SNA at oligonucleotide loading. At DNA loadings exceeding 150 strands per particle, upward shifts in mobility in gel electrophoresis experiments were observed (FIG. 4a), indicating significant size increases in the population, presumably due to SNA aggregation caused by sequence self-complementarity. Evidence of this aggregation is supported by cryogenic transmission electron microscopy (cryoTEM), where a substantial number of particles formed SNA dimers (FIG. 17), which have size difficult to accurately measure by dynamic light scattering (DLS) due to anisotropic shape. Following the addition of exceedingly high equivalents of DNA to liposome (e.g., 10,000 equivalent of DNA/liposome), aggregation of the LSNAs was observed. These aggregates persisted even at elevated temperatures (i.e., 37° C., FIG. 18). SNAs with complementary or self-complementary sequences are known to assemble into aggregates, and various colloidal crystals can be produced with careful thermal treatments and processes, and such polyvalent, hybridization-induced crystallization produces aggregates with higher melting temperatures than the analogous linear ODN duplexes of the same sequence [Mirkin et al., Nature 1996, 382 (6592), 607; Macfarlane et al., Science 2011, 334 (6053), 204; Jones et al., Science 2015, 347 (6224), 1260901; Cutler et al., J. Am. Chem. Soc. 2012, 134 (3), 1376; Laramy et al., ACS Nano 2019, 13 (2), 1412; Park et al., Nature 2008, 451 (7178), 553]. The palindrome sequence of CpG-A is self-complementary, and it was found that the aggregate formation of CpG-A SNAs was indeed mainly due to the palindrome portion of the sequence hybridizing with other particles. To confirm this effect was due to self-complementarity, the poly(G) portion was removed from the sequence and it was observed that at high DNA additions, LSNA aggregation still occurred. Conversely, no aggregation was observed when the same amount of poly(G) sequence was added to the 5′- and 3′-ends of a dummy sequence (e.g., T20 sequence), even after storage at 4° C. for several days. Liposomes functionalized with complementary DNA sequences are known to fuse together, aided by DNA hybridization [Ries et al., Org. Biomol. Chem. 2017, 15 (42), 8936]. However, cryoTEM of CpG-A SNA aggregates (FIG. 18) revealed that the SNAs crowd together, yet individual versus fused particles are still distinguishable. This indicated that the poly(G) sequence on the 3′-end may prevent liposome fusion.

### Conclusions

The unique properties of the SNA architecture allow for the delivery of diverse ODN classes at programmable ratios and with high uptake and codelivery to the same target cell. Through generating a series of SNAs incorporating CpG-A and CpG-B on the same nanoparticle scaffold at different ratios, it was determined that the SNA architecture provided high intracellular association of these two distinct ODN classes. Furthermore, codelivery of both CpG classes on the dual-CpG SNA scaffold led to synergistic DC costimulatory molecule activation, where cytokine and IFN secretion is proportional to the fraction of each of their activating CpG classes (CpG-B and CpG-A, respectively). Although the two sequences used in this report are both TLR9 ligands, the high association and intracellular trafficking behaviors are governed by the SNA scaffold and are independent of sequence, as these sequences are trafficked to the same location despite being both physically and chemically diverse. Therefore, these findings should be generalizable to the design and synthesis of SNAs containing any combination of multi-sequence SNAs for desired applications.

Thus, sequence multiplicity spherical nucleic acids (SNAs) are shown herein to provide a power platform for co-delivery of several oligonucleotides with controlled ratios on the same particle to ensure such ratio gets taken up by cells. The ratios of each sequence incorporated can be determined either from experimentation or from rational design. It was shown herein that, regardless of sequence, spherical nucleic acids' intracellular trafficking follows a particular pathway and schedule, unlike their linear counterparts which may traffic very diversely. This can be taken as an advantage of, for example, using a higher fraction of a sequence that binds to a receptor in an organelle where spherical nucleic acid localizes for less time, and vice versa.

### Example 2

Methods. 1×10⁷ human PBMCs were treated with 200 nM human CpG SNAs for 24 hours. Dual-CpG SNA included 70% CpG-A and 30% CpG-B. SNAs in this example were generally synthesized using methods as described in Example 1, above.

Sequences. Human CpG-A: ggGGGACGATCGTCgggggg (SEQ ID NO: 11), human CpG-B: tcgtcgttttgtcgttttgtcgtt (SEQ ID NO: 12) (Bases shown in capital letters are phosphodiester, and those in lower case are phosphorothioate).

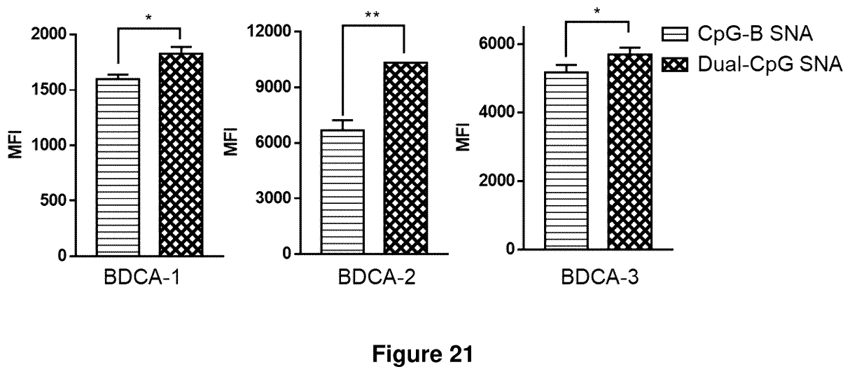

Results/Conclusion. Dual-CpG SNAs promoted significant enhancement in dendritic cell activation (quantified by co-stimulation marker CD80 expression) in all dendritic cell subtypes collected from human peripheral blood mononuclear cells (PBMCs), compared to SNAs containing only CpG-B, demonstrating that the dual-CpG SNAs are translatable to human cells as more potent immunotherapy nanomaterials. See FIG. 21.

What is claimed is:

1. A spherical nucleic acid (SNA) comprising a nanoparticle core and an oligonucleotide shell attached to the external surface of the nanoparticle core, wherein the oligonucleotide shell comprises a mixture of class A CpG oligonucleotides and class B CpG oligonucleotides, wherein the SNA does not comprise a class C CpG oligonucleotide.

2. A spherical nucleic acid (SNA) comprising a nanoparticle core and an oligonucleotide shell attached to the external surface of the nanoparticle core, wherein the oligonucleotide shell consists of a mixture of class A CpG oligonucleotides and class B CpG oligonucleotides.

3. A spherical nucleic acid (SNA) comprising a nanoparticle core and an oligonucleotide shell attached to the external surface of the nanoparticle core, wherein the oligonucleotide shell comprises a mixture of class A CpG oligonucleotides and class B CpG oligonucleotides, wherein the ratio of class A CpG oligonucleotides to class B CpG oligonucleotides in the mixture is about 4:1 to about 1:1.

4. The SNA of any one of claims 1-3, wherein the class A CpG oligonucleotides each comprise (i) an internal palindrome sequence containing a CpG motif and (ii) an at least partially phosphorothioated poly(G) sequence at its 5′ and/or 3′ ends.

5. The SNA of any one of claims 1-4, wherein the class A CpG oligonucleotide is DNA.

6. The SNA of any one of claims 1-5, wherein the class B CpG oligonucleotides each comprise a fully phosphorothioated sequence comprising one or more CpG motifs.

7. The SNA of any one of claims 1-6, wherein the class B CpG oligonucleotide is DNA.

8. The SNA of any one of claims 1-7, wherein the oligonucleotide shell comprises about 5 to about 150 oligonucleotides.

9. The SNA of any one of claims 1-8, wherein the oligonucleotide shell comprises about 5 to about 50 oligonucleotides.

10. The SNA of any one of claims 1-8, wherein the oligonucleotide shell comprises about 75 to about 100 oligonucleotides.

11. The SNA of any one of claims 1-10, wherein the ratio of class A CpG oligonucleotides to class B CpG oligonucleotides in the mixture is about 7:3.

12. The SNA of any one of claim 1-2 or 4-11, wherein the ratio of class A CpG oligonucleotides to class B CpG oligonucleotides in the mixture is about 4:1 to about 1:1.

13. The SNA of any one of claims 1-12, wherein each class A CpG oligonucleotide is about 10 to about 50 nucleotides in length.

14. The SNA of any one of claims 1-13, wherein each class A CpG oligonucleotide is about 15 to about 26 nucleotides in length.

15. The SNA of any one of claims 1-14, wherein each class B CpG oligonucleotide is about 10 to about 50 nucleotides in length.

16. The SNA of any one of claims 1-15, wherein each class B CpG oligonucleotide is about 18 to about 28 nucleotides in length.

17. The SNA of any one of claim 1 or 3-16, further comprising an inhibitory oligonucleotide.

18. The SNA of claim 17, wherein the inhibitory oligonucleotide is an antagonist oligonucleotide, antisense DNA, small interfering RNA (siRNA), an aptamer, a short hairpin RNA (shRNA), a DNAzyme, or an aptazyme.

19. The SNA of any one of claims 1-18, wherein the nanoparticle core is a metallic core, a semiconductor core, an insulator core, an upconverting core, a micellar core, a dendrimer core, a liposomal core, a polymer core, a metal-organic framework core, a protein core, or a combination thereof.

20. The SNA of claim 19, wherein the polymer is polylactide, a polylactide-polyglycolide copolymer, a polycaprolactone, a polyacrylate, alginate, albumin, polypyrrole, polythiophene, polyaniline, polyethylenimine, poly(methyl methacrylate), poly(lactic-co-glycolic acid) (PLGA), or chitosan.

21. The SNA of claim 19, wherein the nanoparticle core is gold, silver, platinum, aluminum, palladium, copper, cobalt, indium, cadmium selenide, iron oxide, fullerene, metal-organic framework, silica, zinc sulfide, or nickel.

22. The SNA of claim 19, wherein the nanoparticle core is a protein core.

23. The SNA of claim 22, wherein the protein core is an enzyme, a therapeutic protein, a structural protein, a defensive protein, a storage protein, a transport protein, a hormone, a receptor protein, a motor protein, or a fluorescent protein.

24. The SNA of any one of claims 1-23, further comprising an additional agent.

25. The SNA of claim 24, wherein the additional agent is a protein, a small molecule, or a peptide, or a combination thereof.

26. The SNA of claim 25, wherein the protein is an antibody, an antigen, a cytokine, a chemokine, an interferon, or a combination thereof.

27. The SNA of any one of claims 1-26, wherein the SNA is from about 1 to about 150 nanometers (nm) in diameter.

28. The SNA of any one of claims 1-27, wherein the oligonucleotide shell comprises about 4 to about 1000 oligonucleotides.

29. A composition comprising a plurality of the spherical nucleic acids (SNAs) of any one of claims 1-28.

30. A method of inhibiting expression of a gene comprising the step of hybridizing a polynucleotide encoding the gene product with the spherical nucleic acid (SNA) of any one of claim 1 or 3-28, or the composition of claim 29, wherein hybridizing between the polynucleotide and one or more oligonucleotides in the oligonucleotide shell occurs over a length of the polynucleotide with a degree of complementarity sufficient to inhibit expression of the gene product.

31. The method of claim 30 wherein expression of the gene product is inhibited in vivo.

32. The method of claim 30 wherein expression of the gene product is inhibited in vitro.

33. A method for up-regulating activity of a toll-like receptor (TLR), comprising contacting a cell having the toll-like receptor with the spherical nucleic acid (SNA) of any one of claims 1-28, or the composition of claim 29.

34. The method of claim 33 wherein the oligonucleotide shell further comprises one or more oligonucleotides that is a TLR agonist.

35. The method of claim 33 or claim 34 wherein the toll-like receptor is chosen from the group consisting of toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, and toll-like receptor 13.

36. A method for down-regulating activity of a toll-like receptor (TLR), comprising contacting a cell having the toll-like receptor with the spherical nucleic acid (SNA) of any one of claims 1-28, or the composition of claim 29.

37. The method of claim 36 wherein the oligonucleotide shell further comprises one or more oligonucleotides that is a TLR antagonist.

38. The method of claim 36 or claim 37 wherein the toll-like receptor is chosen from the group consisting of toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, and toll-like receptor 13.

39. The method of any one of claims 33-38 which is performed in vitro.

40. The method of any one of claims 33-38 which is performed in vivo.

41. A method of treating a disorder comprising administering an effective amount of the SNA of any one of claims 1-28 or the composition of claim 29 to a subject in need thereof, wherein the administering treats the disorder.

42. The method of claim 41, wherein the disorder is cancer, an infectious disease, an autoimmune disease, or a combination thereof.
